(19) United States
(12) Patent Application Publication
(10) Pub. No.: US 2024/0366584 A1
(43) Pub. Date: Nov. 7, 2024

(54) COMBINATION TREATMENT OF CANCER WITH A PARP INHIBITOR AND A LIPOPHILIC STATIN

(71) Applicant: TESARO, INC., Wilmington, DE (US)

(72) Inventor: Giovanna BERGAMINI, Heidelberg (DE)

(21) Appl. No.: 18/567,085

(22) PCT Filed: Jun. 17, 2022
(86) PCT No.: PCT/US2022/033969

§ 371 Date: Dec. 5, 2023

Related U.S. Application Data
(60) Provisional application No. 63/305,907, filed on Feb. 2, 2022.
(60) Provisional application No. 63/212,893, filed on Jun. 21, 2021.

(51) Int. Cl. A61K 31/454; A61K 31/366; A61K 31/405; A61P 35/00

(52) U.S. Cl. CPC A61K 31/454; A61K 31/366; A61K 31/405; A61P 35/00

(57) ABSTRACT

The present invention relates to combinations useful in the treatment of cancer. In particular, the present invention relates to a combination comprising a poly (ADP-ribose) polymerase inhibitor (PARP inhibitor) and a lipophilic statin, for use in the treatment of cancer.

### FIELD OF THE INVENTION

The present invention relates to combinations useful in the treatment of cancer. In particular, the present invention relates to a combination comprising a poly (ADP-ribose) polymerase inhibitor (PARP inhibitor) and a lipophilic statin or an agent that is capable of modulating the cholesterol biosynthesis pathway, for use in the treatment of cancer.

### BACKGROUND TO THE INVENTION

Poly (ADP-ribose) polymerases are a family of enzymes involved in DNA damage repair (DDR) and PARP inhibition leads to accumulation of single-strand breaks (SSBs), which then leads to an accumulation of double strand breaks (DSBs). Cells with an increasing number of DSBs are more dependent on other DNA repair pathways, mainly the homologous recombination (HR) repair pathway. Cancer cells with HR deficiencies are therefore even more susceptible to PARP impairment of the BER pathway. There are currently four approved PARP inhibitors (niraparib, olaparib, rucaparib and talazoparib), with many more currently in clinical trials.

Niraparib is approved as a PARP inhibitor for the maintenance treatment of adult patients with advanced or recurrent epithelial ovarian, fallopian tube, or primary peritoneal cancer who are in a complete or partial response to first-line platinum-based chemotherapy; and for the treatment of adult patients with advanced ovarian, fallopian tube, or primary peritoneal cancer who have been treated with three or more prior chemotherapy regimens and whose cancer is associated with homologous recombination deficiency (HRD) positive status defined by either a deleterious or suspected deleterious BRCA mutation, or genomic instability and who have progressed more than six months after response to the last platinum-based chemotherapy.

Olaparib is approved as a PARP inhibitor for use in the treatment of ovarian cancer, breast cancer, pancreatic cancer and prostate cancer in certain patient subgroups. In ovarian cancer, olaparib is used for the maintenance treatment of adult patients with deleterious or suspected deleterious germline or somatic BRCA-mutated advanced epithelial ovarian, fallopian tube or primary peritoneal cancer who are in complete or partial response to first-line platinum-based chemotherapy; for the maintenance treatment of adult patients with recurrent epithelial ovarian, fallopian tube or primary peritoneal cancer, who are in complete or partial response to platinum-based chemotherapy; and for the treatment of adult patients with deleterious or suspected deleterious germline BRCA-mutated (gBRCAm) advanced ovarian cancer who have been treated with three or more prior lines of chemotherapy.

Rucaparib is approved as PARP inhibitor for use in the treatment of ovarian and prostate cancer in certain patient subgroups. Talazoparib is approved for use in the treatment of breast cancer in adult patients with deleterious or suspected deleterious germline BRCA-mutated HER2-negative locally advanced metastatic breast cancer.

Notably, niraparib is the only PARP inhibitor, and small molecule, approved for front-line maintenance treatment in patients with tumours irrespective of HR status (typically measured as HR deficiency score). In a Phase 3 clinical trial, the PRIMA study, participants with advanced ovarian cancer after having received platinum therapy, the group of participants with low HRD score traditionally not selected for treatment with a PARP inhibitor also showed benefits with niraparib treatment.

Statins, also known as HMG-COA reductase inhibitors, are generally prescribed for the treatment of high cholesterol to help lower the level of low-density lipoprotein (LDL) in the blood. Known statins include atorvastatin (LIPITOR), simvastatin (ZOCOR), lovastatin (MEVACOR), pravastatin (PRAVACHOL), rosuvastatin (CRESTOR), pitavastatin (LIVALO) and fluvastatin (LESCOL).

Statins may be characterised as either hydrophilic or lipophilic. Lipophilic statins are characterised by having a positive log D at pH 7.4, i.e. a log D above zero. For example, atorvastatin, simvastatin, fluvastatin, lovastatin and pitavastatin have been reported as having the following log D values at a pH of 7.4: atorvastatin 1.53, simvastatin 2.44, fluvastatin 1.75, lovastatin 2.59 and pitavastatin 1.50 (Matre et al., Vascular Health and Risk Management, 2016, pages 153-161) and are defined as being lipophilic statins.

In recent years, there has been some discussion on the potential use of statins in the treatment of certain cancers (Di Bello et al., Frontiers in Chemistry, 2020, page 516; Longo et al., Clinical Cancer Research, 2020, page 5791; and Hassanabad, Translational Lung Cancer Research, 2019, pages 692-699).

### SUMMARY OF THE INVENTION

The present inventors have found that the combination of a PARP inhibitor and a lipophilic statin is useful in the treatment of a range of cancer types. In particular, it has been found that the combination of a PARP inhibitor with a lipophilic statin improves median progression free survival (PFS) following treatment, relative to treatment with either agent alone.

The present inventors have also found that a combination of a poly (ADP-ribose) polymerase inhibitor (PARP inhibitor) and an agent that is capable of modulating the cholesterol biosynthesis pathway is useful in the treatment of cancer. For example, statins are known inhibitors of the cholesterol biosynthesis pathway, inhibiting HMG-COA reductase at the beginning of the pathway. Thus, inhibition of HMG-COA reductase using a statin (in particular a lipophilic statin) in combination with a PARP inhibitor is useful in the treatment of cancer.

Also, lanosterol synthase (LSS, known also as oxidosqualene cyclase (OSC)) is involved in cholesterol biosynthesis and it has been found that inhibition of LSS using an LSS inhibitor in combination with a PARP inhibitor is useful in the treatment of cancer.

In a first aspect, the invention relates to a method of treating cancer in a patient in need thereof, the method comprising administering to a patient a therapeutically effective amount of a poly (ADP-ribose) polymerase inhibitor (PARP inhibitor); and a therapeutically effective amount of a lipophilic statin.

In a second aspect, the invention relates to a method of treating cancer in a patient in need thereof, the method comprising administering to the patient a therapeutically effective amount of a poly (ADP-ribose) polymerase inhibitor (PARP inhibitor), wherein the patient is receiving a therapeutically effective amount of a lipophilic statin.

In a third aspect, the invention relates to a combination comprising a poly (ADP-ribose) polymerase inhibitor (PARP inhibitor) and a lipophilic statin, for use in the treatment of cancer.

In a fourth aspect, the invention relates to a PARP inhibitor for use in the treatment of cancer, wherein the PARP inhibitor is administered with a lipophilic statin.

In a fifth aspect, the invention relates to the use of a combination of a poly (ADP-ribose) polymerase inhibitor (PARP inhibitor) and a lipophilic statin in the manufacture of a medicament for the treatment of cancer.

In a sixth aspect, the invention relates to a method of treating cancer in a patient in need thereof, the method comprising administering to the patient a therapeutically effective amount of a poly (ADP-ribose) polymerase inhibitor (PARP inhibitor); and a therapeutically effective amount of an agent capable of modulating the cholesterol biosynthesis pathway.

### DESCRIPTION OF DRAWINGS/FIGURES

FIG. 1 is a graph showing survival probability over time in months for patients on placebo versus statin alone versus niraparib alone versus niraparib in combination with statin treatment.

FIG. 2 is a flowchart illustrating the cholesterol biosynthesis pathway.

FIG. 3 illustrates the results for experiments with niraparib and olaparib as single agent or in combination with LSS inhibitor Ro-48-8071, as described in Example 8.

### DETAILED DESCRIPTION OF THE INVENTION

An aspect of the present invention relates to a method of treating cancer in a patient in need thereof, the method comprising administering to the patient a therapeutically effective amount of a poly (ADP-ribose) polymerase inhibitor (PARP inhibitor); and a therapeutically effective amount of a lipophilic statin.

Another aspect of the present invention relates to a method of treating cancer in a patient in need thereof, the method comprising administering to the patient a therapeutically effective amount of a poly (ADP-ribose) polymerase inhibitor (PARP inhibitor), wherein the patient is receiving a therapeutically effective amount of a lipophilic statin. For example, the patient may be receiving treatment with a lipophilic statin prior to treatment with a PARP inhibitor. The treatment with a lipophilic statin may be due to the patient requiring statin treatment to lower cholesterol, i.e., for the treatment of hypercholesterolemia, hypertriglyceridemia or mixed hyperlipidemia, or for any other indication for which statins are used as a therapy. For example, atherosclerosis and cardiovascular diseases (including coronary heart disease, angina, heart attacks, stroke).

In an embodiment, the invention relates to a method of treating cancer in a patient in need thereof, the method comprising administering to the patient a therapeutically effective amount of a poly (ADP-ribose) polymerase inhibitor (PARP inhibitor), wherein the patient is already receiving a therapeutically effective amount of a lipophilic statin or already on a statin treatment regimen or has a maintained statin concentration in the blood.

In an aspect, the invention relates to a method of treating cancer in a patient in need thereof, the method comprising administering to the patient (i) a therapeutically effective amount of a poly (ADP-ribose) polymerase inhibitor (PARP inhibitor); and (ii) a therapeutically effective amount of an agent capable of modulating the cholesterol biosynthesis pathway.

For example, statins are known inhibitors of the cholesterol biosynthesis pathway, inhibiting HMG-COA reductase at the beginning of the pathway. Thus, inhibition of HMG-CoA reductase using a statin (in particular a lipophilic statin) in combination with a PARP inhibitor is useful in the treatment of cancer.

Also, lanosterol synthase (LSS) is involved in cholesterol biosynthesis and it has been found that inhibition of LSS using an LSS inhibitor in combination with a PARP inhibitor is useful in the treatment of cancer.

Therefore, in an embodiment, the invention relates to a method of treating cancer in a patient in need thereof, the method comprising administering to the patient (i) a therapeutically effective amount of a poly (ADP-ribose) polymerase inhibitor (PARP inhibitor); and (ii) a therapeutically effective amount of an LSS inhibitor.

In an embodiment, there is a combination of a poly (ADP-ribose) polymerase inhibitor (PARP inhibitor) and an agent that is capable of modulating the cholesterol biosynthesis pathway, which is useful in the treatment of cancer.

The use of the term patient is intended to refer to a human patient, wherein the patient is a cancer patient, i.e. a patient having cancer.

In an embodiment, the PARP inhibitor is selected from the group consisting of niraparib, olaparib, talazoparib, rucaparib, veliparib and AZD5305 or a pharmaceutically acceptable salt thereof. The PARP inhibitor may also be selected from the group consisting of THG-008, RBN-2397, TSL-1502, NMS-03305293, HWH-340, STP06-1002, JPI-547, ABT-767, simmiparib, stenoparib, IDX-1197, SC-10914, AMXI-5001, amelparib dihydrochloride dihydrate, CK-102, IMP-4297, pamiparib, and fluzoparib or a pharmaceutically acceptable salt thereof. AZD5305 is described in WO 2021/013735 and is known to be 5-[4-[(7-ethyl-6-oxo-5H-1,5-napthyridine-3-yl)methyl]piperazin-1-yl]-N-methyl-pyridine-2-carboxamide and has the following structure:

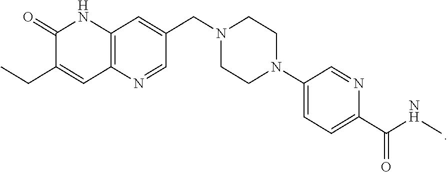 [SMILES: CCc1cc2ncc(CN3CCN(c4ccc(C(=O)NC)nc4)CC3)cc2[nH]c1=O]

In an embodiment, the PARP inhibitor may also be AZD9574 or a pharmaceutically acceptable salt thereof. AZD9574 is described in WO 2021/260092 and is known to be 6-fluoro-5-[4-[(5-fluoro-2-methyl-3-oxo-4H-quinoxalin-6-yl)methyl] piperazin-1-yl]-N-methyl-pyridine-2-carboxamide and has the following structure:

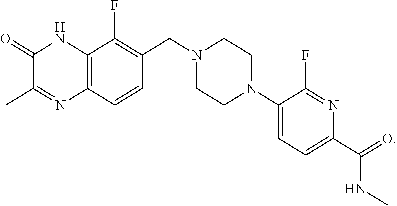 [SMILES: CNC(=O)c1ccc(N2CCN(Cc3ccc4nc(C)c(=O)[nH]c4c3F)CC2)c(F)n1]

Therefore, in an embodiment, the PARP inhibitor is selected from the group consisting of niraparib, olaparib, talazoparib, rucaparib, veliparib, AZD5305 and AZD9574 or a pharmaceutically acceptable salt thereof.

In an embodiment, the PARP inhibitor is selected from the group consisting of niraparib, olaparib, talazoparib and rucaparib or a pharmaceutically acceptable salt thereof.

In a further embodiment, the PARP inhibitor is selected from the group consisting of niraparib, talazoparib and rucaparib or a pharmaceutically acceptable salt thereof.

In an embodiment, the PARP inhibitor may be selective for PARP1 over PARP2.

In an embodiment, the PARP inhibitor is AZD5305 or a pharmaceutically acceptable salt thereof, as defined above.

It should be noted that reference to a PARP inhibitor also includes reference to their pharmaceutically acceptable salts. In other words, “PARP inhibitor” is synonymous with “PARP inhibitor or a pharmaceutically acceptable salt thereof”.

In one embodiment, the PARP inhibitor is niraparib or a pharmaceutically acceptable salt thereof, in particular niraparib tosylate monohydrate. Reference to niraparib is intended to include all versions of niraparib, for example salts including pharmaceutically acceptable salts, polymorphs and solvates, including the tosylate monohydrate.

In an embodiment, the total daily dose of niraparib is 100 mg, 200 mg or 300 mg taken once daily. In a particular embodiment, the total daily dose of niraparib is 200 mg or 300 mg taken once daily. The dose may be provided as 100 mg capsules (in particular, hard capsules) or 100 mg tablets. In an embodiment, each capsule or tablet contains niraparib tosylate monohydrate equivalent to 100 mg niraparib. In an embodiment when niraparib is for use in the treatment of first-line ovarian cancer then the dose is 200 mg taken once daily. However, for patients who weigh≥77 kg and have a baseline platelet count≥150,000/μL then the dose is 300 mg taken once daily. For the treatment of recurrent ovarian cancer or other cancers, the total recommended daily dose is 300 mg taken once daily.

In another embodiment, the PARP inhibitor is olaparib or a pharmaceutically acceptable salt thereof Reference to olaparib is intended to include all versions of olaparib, for example salts including pharmaceutically acceptable salts, polymorphs and solvates.

In an embodiment, the dose of olaparib is 300 mg taken twice daily, equivalent to a total daily dose of 600 mg. The dose may be provided as capsules, in particular hard capsules containing 50 mg of olaparib. Therefore, to achieve a dose of 300 mg it is necessary for a patient to take six capsules. The dose may also be provided as 100 mg or 150 mg tablets, where the tablets may be film-coated.

In another embodiment, the PARP inhibitor is rucaparib or a pharmaceutically acceptable salt thereof, in particular rucaparib camsylate. Reference to rucaparib is intended to include all versions of rucaparib, for example salts including pharmaceutically acceptable salts, polymorphs and solvates.

In an embodiment, the dose of rucaparib is 600 mg taken twice daily, equivalent to a total daily dose of rucaparib of 1200 mg. The dose may be provided as tablets available as 200 mg, 250 mg and 300 mg tablets, which may be film-coated.

In another embodiment, the PARP inhibitor is talazoparib or a pharmaceutically acceptable salt thereof, in particular talazoparib tosylate. Reference to talozaparib is intended to include all versions of talazoparib, for example salts including pharmaceutically acceptable salts, polymorphs and solvates.

In an embodiment, the dose of talazoparib is 1 mg taken once daily. The dose may be provided as capsules containing talazoparib tosylate equivalent to 0.25 mg or 1 mg talazoparib.

In an embodiment, the PARP inhibitor is administered daily.

The PARP inhibitors may be administered to a patient by any convenient route of administration, whether systemically/peripherally or at the site of desired action, including but not limited to, oral (e.g. by ingestion); pulmonary (e.g. by inhalation or insufflation therapy 20 using, e.g. an aerosol, e.g. through mouth or nose); and parenteral, (e.g. by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal).

In an embodiment, the PARP inhibitor is administered orally. For example, by capsule or tablet, or by liquid form.

In one embodiment, in particular when the PARP inhibitor is niraparib, it may be administered orally by capsule or tablet. In one embodiment, niraparib is administered by capsule. In another embodiment, niraparib is administered by tablet.

In another embodiment, the PARP inhibitor is administered orally, by way of liquid form. For example, as a suspension. In some embodiments it will be suitable to administer the PARP inhibitor in liquid form, for example via a tube. In this embodiment, it may also be suitable to administer the lipophilic statin by way of liquid form.

In an embodiment, the lipophilic statin is defined by having a Log D value of >0 at a pH of 7.4 or >0 at a pH of 7.0. These Log D values are well known in the literature for characterizing statins, for example as reported in Shitara et al., Pharmacology & Therapeutics, 2006, pages 71-105. For example, atorvastatin, simvastatin, fluvastatin, lovastatin, cerivastatin and pitavastatin have been reported as having the following log D values at a pH of 7.0: atorvastatin 1.53, simvastatin 4.4, fluvastatin 1.75, lovastatin 3.91, cerivastatin 2.32 and pitavastatin 1.50. Higher numbers indicate increased lipophilicity. For completeness, in the same reference, values are also reported for pravastatin and rosuvastatin of −0.47 (at pH 7.0) and −0.25 to −0.50 (at pH 7.4), respectively. Pravastatin and rosuvastatin are indicated as being hydrophilic statins in Matre et al., Vascular Health and Risk Management, 2016, pages 153-161. Log D values may be determined by any known method known to a person skilled in the art, for example as described in onlinelibrary.wiley.com/doi/pdl/10.1002/3527601473.ch2 and Scherrer et al., Journal of Medicinal Chemistry, 1977, 20, pages 53-58.

In an embodiment, the lipophilic statin is selected from the group consisting of atorvastatin, simvastatin, fluvastatin, lovastatin, pitavastatin and cerivastatin, in particular atorvastatin, simvastatin, fluvastatin.

In an embodiment, the lipophilic statin is atorvastatin or simvastatin.

In an embodiment, the lipophilic statin has a Log D of >1.

The guidelines for prescription of statins are as follows: by LDL values 190 mg/dl and higher (40-75 age), or 70 mg/dl or higher in case of diabetes, or selected individuals with 10 years ASCVD risk (AtheroSclerotic CardioVascular Disease) of 7.5% or higher. For example, a patient requiring treatment with a statin may be characterized in this way.

Particular dosage forms and dosing regimens/schedules for approved statin monotherapies are well known to a person skilled in the art.

Statins are also known as HMG-COA reductase inhibitors. Therefore, also contemplated herein is the combination of a PARP inhibitor and an HMG-COA reductase inhibitor.

The PARP inhibitor and lipophilic statin may be administered simultaneously, separately or sequentially. It is to be understood that simultaneous administration means that the PARP inhibitor and statin are administered at the same time, whereas sequential administration means that the PARP inhibitor and statin are administered one after the other, in either order. Separate administration is to be understood as meaning that the PARP inhibitor and statin are administered separately, e.g. at separate times of day, or on different days and according to different treatment regimens. It is also to be understood that the PARP inhibitor and lipophilic statin may be concurrently prescribed to a patient.

In an embodiment, the lipophilic statin is administered before the PARP inhibitor.

In an embodiment, the lipophilic statin is administered after the PARP inhibitor.

In an embodiment, the lipophilic statin is co-administered with the PARP inhibitor.

In an embodiment, a patient is on treatment with a lipophilic statin and the patient is subsequently treated with a PARP inhibitor, for the treatment of cancer.

In an embodiment, a patient is treated for cancer with a PARP inhibitor and is subsequently treated with a lipophilic statin.

In an embodiment, a patient is on treatment with a lipophilic statin and the patient is subsequently treated with niraparib, for the treatment of cancer.

In an embodiment, a patient is treated for cancer with niraparib and is subsequently treated with a lipophilic statin.

The PARP inhibitor and lipophilic statin may be formulated together into a single pharmaceutical composition (e.g. tablet or capsule), or may be individually formulated in separate pharmaceutical compositions. In one embodiment, each therapeutic agent in the combination is individually formulated into its own pharmaceutical composition and each of the pharmaceutical compositions are administered to treat cancer. In this embodiment, each of the pharmaceutical compositions may have the same or different carriers, diluents or excipients. The carrier(s), diluent(s) or excipient(s) must be acceptable in the sense of being compatible with the other ingredients of the formulation, capable of pharmaceutical formulation, and not deleterious to the recipient thereof. For example, in one embodiment, a first pharmaceutical composition contains a PARP inhibitor, a second pharmaceutical composition contains a lipophilic statin, and the first and second pharmaceutical compositions are both administered to treat cancer.

In one embodiment, each therapeutic agent in the combination is formulated together into a single pharmaceutical composition and administered to treat cancer. For example, in one embodiment, a single pharmaceutical composition contains both a PARP inhibitor and a lipophilic statin and is administered as a single pharmaceutical composition to treat cancer.

In an embodiment, the combination of the invention may be used in combination with a further therapeutically active agent or agents known to be useful for the treatment of cancer, including immunotherapy (e.g. immune checkpoint inhibitor), cell and gene therapy, chemotherapy or radiation treatment. The term further therapeutically active agent or agents, as used herein, includes any compound or therapeutic agent known to or that demonstrates advantageous properties when administered to a patient in need of treatment for cancer. Furthermore, it does not matter if the compounds are administered in the same dosage form, e.g. one compound may be administered by injection and another compound may be administered orally. Preferably, at least the PARP inhibitor and the statin are administered orally, but the further therapeutically active agent or agents may be administered by any known dosage form.

Typically, any anti-neoplastic agent that has activity versus a susceptible tumor being treated may be co-administered in the treatment of cancer in the present invention. Co-administration is defined as including administration with a further agent or agents. Such further active agent or agents may be selected from any known therapies for the treatment of cancer, including small molecules therapies, antibody therapies, antibody drug conjugate (ADC) therapies and cell & gene therapies. Examples of anti-neoplastic agents include, but are not limited to, chemotherapeutic agents, immune-modulators and immunostimulatory adjuvants. Examples of such agents can be found in Cancer Principles and Practice of Oncology by V. T. Devita, T. S. Lawrence, and S. A. Rosenberg (editors), 11^{th} edition (Nov. 29, 2018), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents; platinum coordination complexes; alkylating agents; antibiotic agents; topoisomerase I inhibitors; topoisomerase II inhibitors; antimetabolites; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; and cancer gene therapy agents.

In an embodiment, the particular further active agent could be selected from one as described in, for example WO 2018/208968, WO 2018/213732 and WO 2020/051142. For example, a PD-1 inhibitor such as dostarlimab or pembrolizumab.

As used herein, the term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011 (see wiley.com/WileyCDA/WileyTitle/productCd-3906390519.html).

Suitable pharmaceutically acceptable salts can include acid or base addition salts.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

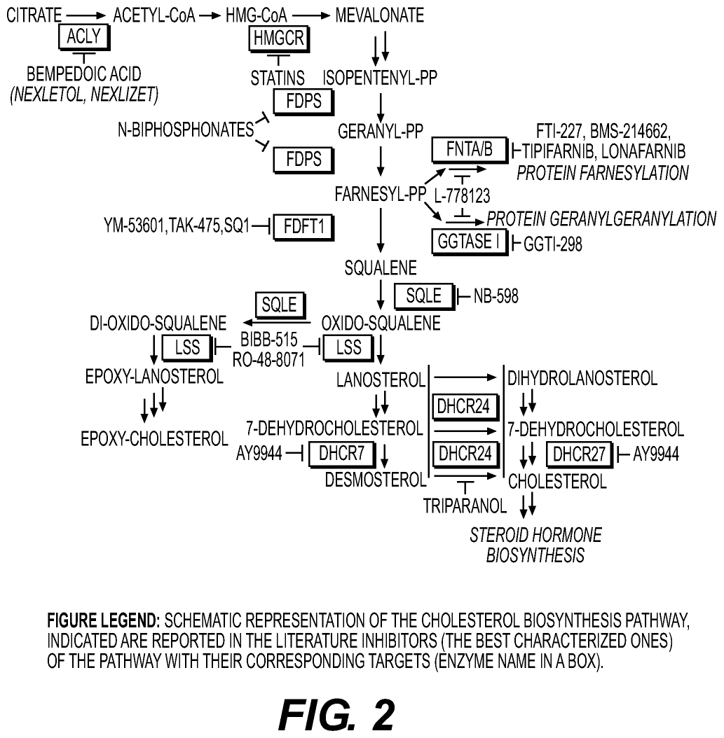

An agent that is capable of modulating the cholesterol biosynthesis pathway may also be referenced as an agent which is an inhibitor of any step in the cholesterol biosynthesis pathway, i.e. a cholesterol biosynthesis inhibitor (for example as shown in FIG. 2). Such agents include, but are not limited to, an HMG-COA reductase inhibitor (i.e. a statin, in particular a lipophilic statin as previously defined), an LSS inhibitor (e.g. Ro 48-8071, BIBB-515 and MI-2), farnesyl pyrophosphate synthase (FDPS) inhibitors from N-BP (N-bisphosphonates) (e.g. pamidronate, alendronate, ibandronate, riserdronate (Actonel, Atelvia), zoledronic acid (Alendronate, Fosamax) and minodronic acid (Recalbon, Bonoteo)), ATP-citrate lyase (ACLY) inhibitors (e.g. bempedoic acid (Nexletol, Nexlizet)), farnesyltransferase (FTase/FNTA, FNTB) inhibitors (FTI) (e.g. L-778123 (Merck), BMS-214662 (Bristol Meyers-Squibb), lonafarnib (Schering-Plough) and tipifarnib/R-115777 (Janssen)), geranylgeranyl transferase (GGTase) inhibitors (e.g. GGTI-298), squalene synthase (FDFT1) inhibitors (e.g. TAK-475 (Lapaquistat), SQ1 and YM-53601), squalene monooxygenase (SQLE) inhibitors (e.g. NB-598), delta (24)-sterol reductase (DHCR24) inhibitors (e.g. triparanol), 7-dehydrocholesterol reductase (DHCR7) inhibitors (e.g. AY9944).

In an embodiment, the agent that is capable of modulating the cholesterol biosynthesis pathway or inhibitor of cholesterol biosynthesis is selected from the group consisting of an HMG-CoA reductase inhibitor (i.e. a statin, in particular a lipophilic statin as previously defined), farnesyl pyrophosphate synthase (FDPS) inhibitors from N-BP (N-bisphosphonates) (e.g. pamidronate, alendronate, ibandronate, riserdronate (Actonel, Atelvia), zoledronic acid (Alendronate, Fosamax) and minodronic acid (Recalbon, Bonoteo)) and ATP-citrate lyase (ACLY) inhibitors (e.g. bempedoic acid (Nexletol, Nexlizet)).

In an embodiment, the agent that is capable of modulating the cholesterol biosynthesis pathway pathway or inhibitor of cholesterol biosynthesis is a lipophilic statin or an LSS inhibitor, particularly a lipophilic statin.

### Statement of Use

The invention relates to a method of treating cancer in a patient in need thereof, the method comprising administering to the patient (i) a therapeutically effective amount of a PARP inhibitor; and (ii) a therapeutically effective amount of a lipophilic statin.

As used herein, the term “therapeutically effective amount” refers to the quantity of a compound (in this context a compound is meant to refer to a PARP inhibitor or lipophilic statin), or a pharmaceutically acceptable salt thereof, which will elicit the desired biological response in an animal or human body.

The invention also relates to a combination comprising a PARP inhibitor and a lipophilic statin, for use in the treatment of cancer.

The invention also relates to use of a combination of a PARP inhibitor and a lipophilic statin in the manufacture of a medicament for the treatment of cancer.

The invention also relates to a PARP inhibitor for use in the treatment of cancer, wherein the PARP inhibitor is administered with a lipophilic statin.

In an embodiment, there is provided a method of treating cancer in a patient in need thereof, the method comprising administering to the patient (i) a therapeutically effective amount of niraparib or a pharmaceutically acceptable salt thereof; and (ii) a therapeutically effective amount of a lipophilic statin.

The invention also relates to a combination comprising niraparib or a pharmaceutically acceptable salt thereof and a lipophilic statin, for use in the treatment of cancer.

The invention also relates to use of a combination of a niraparib or a pharmaceutically acceptable salt thereof and a lipophilic statin in the manufacture of a medicament for the treatment of cancer.

The invention also relates to niraparib for use in the treatment of cancer, wherein niraparib is administered with a lipophilic statin.

In an embodiment, there is provided a method of treating cancer in a patient in need thereof, the method comprising administering to the patient (i) a therapeutically effective amount of AZD5305 or a pharmaceutically acceptable salt thereof; and (ii) a therapeutically effective amount of a lipophilic statin.

The invention also relates to a combination comprising AZD5305 or a pharmaceutically acceptable salt thereof and a lipophilic statin, for use in the treatment of cancer.

The invention also relates to use of a combination of a AZD5305 or a pharmaceutically acceptable salt thereof and a lipophilic statin in the manufacture of a medicament for the treatment of cancer.

The invention also relates to AZD5305 for use in the treatment of cancer, wherein AZD5305 is administered with a lipophilic statin.

In an embodiment, there is provided a method of treating cancer in a patient in need thereof, the method comprising administering to the patient (i) a therapeutically effective amount of AZD9574 or a pharmaceutically acceptable salt thereof; and (ii) a therapeutically effective amount of a lipophilic statin.

The invention also relates to a combination comprising AZD9574 or a pharmaceutically acceptable salt thereof and a lipophilic statin, for use in the treatment of cancer.

The invention also relates to use of a combination of a AZD9574 or a pharmaceutically acceptable salt thereof and a lipophilic statin in the manufacture of a medicament for the treatment of cancer.

The invention also relates to AZD9574 for use in the treatment of cancer, wherein AZD9574 is administered with a lipophilic statin.

As noted above, in an embodiment, the invention relates to a method of treating cancer in a patient in need thereof, the method comprising administering to the patient (i) a therapeutically effective amount of a poly (ADP-ribose) polymerase inhibitor (PARP inhibitor); and (ii) a therapeutically effective amount of an agent capable of modulating the cholesterol biosynthesis pathway. In an embodiment, in this method of treating cancer, the cancer is HR proficient. In other words, the patient may be characterised by having a cancer which is HR proficient.

In an embodiment, the invention relates to a method of treating cancer in a patient in need thereof, the method comprising administering to the patient (i) a therapeutically effective amount of a poly (ADP-ribose) polymerase inhibitor (PARP inhibitor); and (ii) a therapeutically effective amount of an LSS inhibitor (an inhibitor of lanosterol synthase, an enzyme involved in the cholesterol biosynthesis pathway). In an embodiment, in this method of treating cancer, the cancer is HR proficient. In other words, the patient may be characterised by having a cancer which is HR proficient.

In an embodiment, is a combination of a poly (ADP-ribose) polymerase inhibitor (PARP inhibitor) and an agent that is capable of modulating the cholesterol biosynthesis pathway, which is useful in the treatment of cancer.

In an embodiment, the agent that is capable of modulating the cholesterol biosynthesis pathway is an LSS inhibitor. Therefore, also contemplated is a combination of a poly (ADP-ribose) polymerase inhibitor (PARP inhibitor) and an LSS inhibitor, which is useful in the treatment of cancer.

In an embodiment, the agent that is capable of modulating the cholesterol biosynthesis pathway is a lipophilic statin.

In an embodiment, also contemplated is the use of a poly (ADP-ribose) polymerase inhibitor (PARP inhibitor) and an agent that is capable of modulating the cholesterol biosynthesis pathway in the manufacture of a medicament for the treatment of cancer.

In an embodiment, also contemplated is a poly (ADP-ribose) polymerase inhibitor (PARP inhibitor) for use in the treatment of cancer, wherein the poly (ADP-ribose) polymerase inhibitor (PARP inhibitor) is administered with an agent that is capable of modulating the cholesterol biosynthesis pathway, such as an LSS inhibitor.

In an embodiment, the invention relates to a method of treating cancer in a patient in need thereof, the method comprising administering to the patient (i) a therapeutically effective amount of a poly (ADP-ribose) polymerase inhibitor (PARP inhibitor) selected from the group consisting of niraparib, olaparib, AZD9574 and AZD5305; and (ii) a therapeutically effective amount of an agent capable of modulating the cholesterol biosynthesis pathway.

In an embodiment, the invention relates to a method of treating cancer in a patient in need thereof, the method comprising administering to the patient (i) a therapeutically effective amount of a poly (ADP-ribose) polymerase inhibitor (PARP inhibitor) selected from the group consisting of niraparib, olaparib and AZD5305; and (ii) a therapeutically effective amount of an agent capable of modulating the cholesterol biosynthesis pathway.

In an embodiment, also contemplated is a method of treating cancer in a patient in need thereof, the method comprising administering to the patient (i) a therapeutically effective amount of a poly (ADP-ribose) polymerase inhibitor (PARP inhibitor) selected from the group consisting of niraparib, olaparib, AZD9574 and AZD5305; and (ii) a therapeutically effective amount of an LSS inhibitor (an inhibitor of lanosterol synthase, an enzyme involved in the cholesterol biosynthesis pathway). In an embodiment, in this method of treating cancer, the cancer is HR proficient. In other words, the patient may be characterised by having a cancer which is HR proficient.

In an embodiment, also contemplated is a method of treating cancer in a patient in need thereof, the method comprising administering to the patient (i) a therapeutically effective amount of a poly (ADP-ribose) polymerase inhibitor (PARP inhibitor) selected from the group consisting of niraparib, olaparib and AZD5305; and (ii) a therapeutically effective amount of an LSS inhibitor (an inhibitor of lanosterol synthase, an enzyme involved in the cholesterol biosynthesis pathway). In an embodiment, in this method of treating cancer, the cancer is HR proficient. In other words, the patient may be characterised by having a cancer which is HR proficient.

In an embodiment, also contemplated is a combination of a poly (ADP-ribose) polymerase inhibitor (PARP inhibitor) selected from the group consisting of niraparib, olaparib, AZD9574 and AZD5305; and an agent that is capable of modulating the cholesterol biosynthesis pathway, which is useful in the treatment of cancer.

In an embodiment, also contemplated is a combination of a poly (ADP-ribose) polymerase inhibitor (PARP inhibitor) selected from the group consisting of niraparib, olaparib and AZD5305; and an agent that is capable of modulating the cholesterol biosynthesis pathway, which is useful in the treatment of cancer.

In an embodiment, the agent that is capable of modulating the cholesterol biosynthesis pathway is an LSS inhibitor.

Therefore, also contemplated is a combination of a poly (ADP-ribose) polymerase inhibitor (PARP inhibitor) selected from the group consisting of niraparib, olaparib, AZD9574 and AZD5305; and an LSS inhibitor, which is useful in the treatment of cancer.

Therefore, also contemplated is a combination of a poly (ADP-ribose) polymerase inhibitor (PARP inhibitor) selected from the group consisting of niraparib, olaparib and AZD5305; and an LSS inhibitor, which is useful in the treatment of cancer.

In an embodiment, also contemplated is the use of a a poly (ADP-ribose) polymerase inhibitor (PARP inhibitor) selected from the group consisting of niraparib, olaparib, AZD9574 and AZD5305; and an agent that is capable of modulating the cholesterol biosynthesis pathway in the manufacture of a medicament for the treatment of cancer.

In an embodiment, also contemplated is the use of a a poly (ADP-ribose) polymerase inhibitor (PARP inhibitor) selected from the group consisting of niraparib, olaparib and AZD5305; and an agent that is capable of modulating the cholesterol biosynthesis pathway in the manufacture of a medicament for the treatment of cancer.

In an embodiment, also contemplated is a poly (ADP-ribose) polymerase inhibitor (PARP inhibitor) selected from the group consisting of niraparib, olaparib, AZD9574 and AZD5305, for use in the treatment of cancer, wherein the poly (ADP-ribose) polymerase inhibitor (PARP inhibitor) is administered with an agent that is capable of modulating the cholesterol biosynthesis pathway, such as an LSS inhibitor.

In an embodiment, also contemplated is a poly (ADP-ribose) polymerase inhibitor (PARP inhibitor) selected from the group consisting of niraparib, olaparib and AZD5305, for use in the treatment of cancer, wherein the poly (ADP-ribose) polymerase inhibitor (PARP inhibitor) is administered with an agent that is capable of modulating the cholesterol biosynthesis pathway, such as an LSS inhibitor.

In an embodiment, the invention relates to a method of treating cancer in a patient in need thereof, the method comprising administering to the patient (i) a therapeutically effective amount of niraparib; and (ii) a therapeutically effective amount of an agent capable of modulating the cholesterol biosynthesis pathway.

In an embodiment, also contemplated is a method of treating cancer in a patient in need thereof, the method comprising administering to the patient (i) a therapeutically effective amount of niraparib; and (ii) a therapeutically effective amount of an LSS inhibitor (an inhibitor of lanosterol synthase, an enzyme involved in the cholesterol biosynthesis pathway). In an embodiment, in this method of treating cancer, the cancer is HR proficient. In other words, the patient may be characterised by having a cancer which is HR proficient.

In an embodiment, also contemplated is a combination of niraparib; and an agent that is capable of modulating the cholesterol biosynthesis pathway, which is useful in the treatment of cancer.

In an embodiment, the agent that is capable of modulating the cholesterol biosynthesis pathway is an LSS inhibitor.

Therefore, also contemplated is a combination of niraparib; and an LSS inhibitor, which is useful in the treatment of cancer.

In an embodiment, also contemplated is the use of niraparib; and an agent that is capable of modulating the cholesterol biosynthesis pathway in the manufacture of a medicament for the treatment of cancer.

In an embodiment, also contemplated is niraparib for use in the treatment of cancer, wherein niraparib is administered with an agent that is capable of modulating the cholesterol biosynthesis pathway, such as an LSS inhibitor.

In an embodiment, the invention relates to a method of treating cancer in a patient in need thereof, the method comprising administering to the patient (i) a therapeutically effective amount of AZD5305; and (ii) a therapeutically effective amount of an agent capable of modulating the cholesterol biosynthesis pathway.

In an embodiment, also contemplated is a method of treating cancer in a patient in need thereof, the method comprising administering to the patient (i) a therapeutically effective amount AZD5305; and (ii) a therapeutically effective amount of an LSS inhibitor (an inhibitor of lanosterol synthase, an enzyme involved in the cholesterol biosynthesis pathway). In an embodiment, in this method of treating cancer, the cancer is HR proficient. In other words, the patient may be characterised by having a cancer which is HR proficient.

In an embodiment, also contemplated is a combination of AZD5305; and an agent that is capable of modulating the cholesterol biosynthesis pathway, which is useful in the treatment of cancer.

In an embodiment, the agent that is capable of modulating the cholesterol biosynthesis pathway is an LSS inhibitor.

Therefore, also contemplated is a combination of AZD5305; and an LSS inhibitor, which is useful in the treatment of cancer.

In an embodiment, also contemplated is the use of AZD5305; and an agent that is capable of modulating the cholesterol biosynthesis pathway in the manufacture of a medicament for the treatment of cancer.

In an embodiment, also contemplated is AZD5305, for use in the treatment of cancer, wherein AZD5305 is administered with an agent that is capable of modulating the cholesterol biosynthesis pathway, such as an LSS inhibitor.

In an embodiment, the invention relates to a method of treating cancer in a patient in need thereof, the method comprising administering to the patient (i) a therapeutically effective amount of AZD9574; and (ii) a therapeutically effective amount of an agent capable of modulating the cholesterol biosynthesis pathway.

In an embodiment, also contemplated is a method of treating cancer in a patient in need thereof, the method comprising administering to the patient (i) a therapeutically effective amount AZD9574; and (ii) a therapeutically effective amount of an LSS inhibitor (an inhibitor of lanosterol synthase, an enzyme involved in the cholesterol biosynthesis pathway). In an embodiment, in this method of treating cancer, the cancer is HR proficient. In other words, the patient may be characterised by having a cancer which is HR proficient.

In an embodiment, also contemplated is a combination of AZD9574; and an agent that is capable of modulating the cholesterol biosynthesis pathway, which is useful in the treatment of cancer.

In an embodiment, the agent that is capable of modulating the cholesterol biosynthesis pathway is an LSS inhibitor.

Therefore, also contemplated is a combination of AZD9574; and an LSS inhibitor, which is useful in the treatment of cancer.

In an embodiment, also contemplated is the use of AZD9574; and an agent that is capable of modulating the cholesterol biosynthesis pathway in the manufacture of a medicament for the treatment of cancer.

In an embodiment, also contemplated is AZD9574, for use in the treatment of cancer, wherein AZD9574 is administered with an agent that is capable of modulating the cholesterol biosynthesis pathway, such as an LSS inhibitor.

The invention also relates to a method of treating cancer in a patient in need thereof, the method comprising administering to the patient a therapeutically effective amount of a poly (ADP-ribose) polymerase inhibitor (PARP inhibitor), wherein the patient is receiving a therapeutically effective amount of a lipophilic statin. In this embodiment, the method of the invention also comprises treatment of a patient already on statin treatment for a non-cancer related indication in combination with a PARP inhibitor. In another embodiment, the patient is receiving statin for the prevention or treatment of a cancer-related indication.

The invention also relates to a combination comprising a PARP inhibitor and a lipophilic statin, for use in the treatment of cancer, wherein the patient to be treated for cancer is already receiving statin treatment for the treatment of a non-cancer related indication.

In an embodiment, the invention relates to a PARP inhibitor for use in the treatment of cancer, wherein the PARP inhibitor is administered to a patient who is already receiving treatment with a statin.

In an embodiment, the invention is useful in the prevention of cancer. In other words, also contemplated is a method of preventing cancer in a patient in need thereof, the method comprising administering to the patient a therapeutically effective amount of a PARP inhibitor, wherein the patient is receiving a therapeutically effective amount of a lipophilic statin.

The invention also relates to the use of a combination of a PARP inhibitor and a lipophilic statin in the manufacture of a medicament for the treatment of cancer, wherein the patient to be treated for cancer is already receiving statin treatment for the treatment of a non-cancer related indication.

In an embodiment, the invention relates to a combination comprising a PARP inhibitor and a lipophilic statin, for use in the treatment of cancer, wherein the patient to be treated for cancer is receiving treatment for cancer with a PARP inhibitor and is subsequently prescribed a lipophilic statin.

The invention also relates to a method of treating cancer in a patient in need thereof, the method comprising administering to the patient a therapeutically effective amount of a poly (ADP-ribose) polymerase inhibitor (PARP inhibitor), wherein the patient is receiving a therapeutically effective amount of an agent capable of modulating the cholesterol biosynthesis pathway. In this embodiment, the method of the invention also comprises treatment of a patient already on treatment with an agent capable of modulating the cholesterol biosynthesis pathway for a non-cancer related indication in combination with a PARP inhibitor.

The invention also relates to a combination comprising a PARP inhibitor and an agent capable of modulating the cholesterol biosynthesis pathway, for use in the treatment of cancer, wherein the patient to be treated for cancer is already receiving treatment with an agent capable of modulating the cholesterol biosynthesis pathway for the treatment of a non-cancer related indication.

In an embodiment, the invention relates to a PARP inhibitor for use in the treatment of cancer, wherein the PARP inhibitor is administered to a patient who is already receiving treatment with an agent capable of modulating the cholesterol biosynthesis pathway.

The invention also relates to the use of a combination of a PARP inhibitor and an agent capable of modulating the cholesterol biosynthesis pathway in the manufacture of a medicament for the treatment of cancer, wherein the patient to be treated for cancer is already receiving treatment with an agent capable of modulating the cholesterol biosynthesis pathway for the treatment of a non-cancer related indication.

In an embodiment, the invention relates to a combination comprising a PARP inhibitor and an agent capable of modulating the cholesterol biosynthesis pathway, for use in the treatment of cancer, wherein the patient to be treated for cancer is receiving treatment for cancer with a PARP inhibitor and is subsequently prescribed an agent capable of modulating the cholesterol biosynthesis pathway.

The invention also relates to a method of treating cancer in a patient in need thereof, the method comprising administering to the patient a therapeutically effective amount of niraparib, wherein the patient is receiving a therapeutically effective amount of a lipophilic statin. In other words, the method of the invention also comprises treatment of a patient already on statin treatment for a non-cancer related indication in combination with niraparib.

The invention also relates to a combination comprising niraparib and a lipophilic statin, for use in the treatment of cancer, wherein the patient to be treated for cancer is already receiving statin treatment for the treatment of a non-cancer related indication.

In an embodiment, the invention relates to a combination comprising niraparib and a lipophilic statin, for use in the treatment of cancer, wherein the patient to be treated for cancer is receiving treatment for cancer with niraparib and is subsequently prescribed a lipophilic statin.

In an embodiment, the invention relates to niraparib for use in the treatment of cancer, wherein niraparib is administered to a patient already receiving treatment with a statin.

The invention also relates to the use of a combination of niraparib and a lipophilic statin in the manufacture of a medicament for the treatment of cancer, wherein the patient to be treated for cancer is already receiving statin treatment for the treatment of a non-cancer related indication.

The invention also relates to a method of treating cancer in a patient in need thereof, the method comprising administering to the patient a therapeutically effective amount of niraparib, wherein the patient is receiving a therapeutically effective amount of an agent capable of modulating the cholesterol biosynthesis pathway. In this embodiment, the method of the invention also comprises treatment of a patient already on treatment with an agent capable of modulating the cholesterol biosynthesis pathway for a non-cancer related indication in combination with niraparib.

The invention also relates to a combination comprising niraparib and an agent capable of modulating the cholesterol biosynthesis pathway, for use in the treatment of cancer, wherein the patient to be treated for cancer is already receiving treatment with an agent capable of modulating the cholesterol biosynthesis pathway for the treatment of a non-cancer related indication.

In an embodiment, the invention relates to niraparib for use in the treatment of cancer, wherein the niraparib is administered to a patient who is already receiving treatment with an agent capable of modulating the cholesterol biosynthesis pathway.

The invention also relates to the use of a combination of niraparib and an agent capable of modulating the cholesterol biosynthesis pathway in the manufacture of a medicament for the treatment of cancer, wherein the patient to be treated for cancer is already receiving treatment with an agent capable of modulating the cholesterol biosynthesis pathway for the treatment of a non-cancer related indication.

In an embodiment, the invention relates to a combination comprising niraparib and an agent capable of modulating the cholesterol biosynthesis pathway, for use in the treatment of cancer, wherein the patient to be treated for cancer is receiving treatment for cancer with niraparib and is subsequently prescribed an agent capable of modulating the cholesterol biosynthesis pathway.

The invention also relates to a method of treating cancer in a patient in need thereof, the method comprising administering to the patient a therapeutically effective amount of AZD5305, wherein the patient is receiving a therapeutically effective amount of a lipophilic statin. In other words, the method of the invention also comprises treatment of a patient already on statin treatment for a non-cancer related indication in combination with AZD5305.

The invention also relates to a combination comprising AZD5305 and a lipophilic statin, for use in the treatment of cancer, wherein the patient to be treated for cancer is already receiving statin treatment for the treatment of a non-cancer related indication.

In an embodiment, the invention relates to a combination comprising AZD5305 and a lipophilic statin, for use in the treatment of cancer, wherein the patient to be treated for cancer is receiving treatment for cancer with AZD5305 and is subsequently prescribed a lipophilic statin.

In an embodiment, the invention relates to AZD5305 for use in the treatment of cancer, wherein AZD5305 is administered to a patient already receiving treatment with a statin.

The invention also relates to the use of a combination of AZD5305 and a lipophilic statin in the manufacture of a medicament for the treatment of cancer, wherein the patient to be treated for cancer is already receiving statin treatment for the treatment of a non-cancer related indication.

The invention also relates to a method of treating cancer in a patient in need thereof, the method comprising administering to the patient a therapeutically effective amount of AZD5305, wherein the patient is receiving a therapeutically effective amount of an agent capable of modulating the cholesterol biosynthesis pathway. In this embodiment, the method of the invention also comprises treatment of a patient already on treatment with an agent capable of modulating the cholesterol biosynthesis pathway for a non-cancer related indication in combination with AZD5305.

The invention also relates to a combination comprising AZD5305 and an agent capable of modulating the cholesterol biosynthesis pathway, for use in the treatment of cancer, wherein the patient to be treated for cancer is already receiving treatment with an agent capable of modulating the cholesterol biosynthesis pathway for the treatment of a non-cancer related indication.

In an embodiment, the invention relates to AZD5305 for use in the treatment of cancer, wherein the AZD5305 is administered to a patient who is already receiving treatment with an agent capable of modulating the cholesterol biosynthesis pathway.

The invention also relates to the use of a combination of AZD5305 and an agent capable of modulating the cholesterol biosynthesis pathway in the manufacture of a medicament for the treatment of cancer, wherein the patient to be treated for cancer is already receiving treatment with an agent capable of modulating the cholesterol biosynthesis pathway for the treatment of a non-cancer related indication.

In an embodiment, the invention relates to a combination comprising AZD5305 and an agent capable of modulating the cholesterol biosynthesis pathway, for use in the treatment of cancer, wherein the patient to be treated for cancer is receiving treatment for cancer with AZD5305 and is subsequently prescribed an agent capable of modulating the cholesterol biosynthesis pathway.

The invention also relates to a method of treating cancer in a patient in need thereof, the method comprising administering to the patient a therapeutically effective amount of AZD9574, wherein the patient is receiving a therapeutically effective amount of a lipophilic statin. In other words, the method of the invention also comprises treatment of a patient already on statin treatment for a non-cancer related indication in combination with AZD9574.

The invention also relates to a combination comprising AZD9574 and a lipophilic statin, for use in the treatment of cancer, wherein the patient to be treated for cancer is already receiving statin treatment for the treatment of a non-cancer related indication.

In an embodiment, the invention relates to a combination comprising AZD9574 and a lipophilic statin, for use in the treatment of cancer, wherein the patient to be treated for cancer is receiving treatment for cancer with AZD9574 and is subsequently prescribed a lipophilic statin.

In an embodiment, the invention relates to AZD9574 for use in the treatment of cancer, wherein AZD9574 is administered to a patient already receiving treatment with a statin.

The invention also relates to the use of a combination of AZD9574 and a lipophilic statin in the manufacture of a medicament for the treatment of cancer, wherein the patient to be treated for cancer is already receiving statin treatment for the treatment of a non-cancer related indication.

The invention also relates to a method of treating cancer in a patient in need thereof, the method comprising administering to the patient a therapeutically effective amount of AZD9574, wherein the patient is receiving a therapeutically effective amount of an agent capable of modulating the cholesterol biosynthesis pathway. In this embodiment, the method of the invention also comprises treatment of a patient already on treatment with an agent capable of modulating the cholesterol biosynthesis pathway for a non-cancer related indication in combination with AZD9574.

The invention also relates to a combination comprising AZD9574 and an agent capable of modulating the cholesterol biosynthesis pathway, for use in the treatment of cancer, wherein the patient to be treated for cancer is already receiving treatment with an agent capable of modulating the cholesterol biosynthesis pathway for the treatment of a non-cancer related indication.

In an embodiment, the invention relates to AZD9574 for use in the treatment of cancer, wherein the AZD9574 is administered to a patient who is already receiving treatment with an agent capable of modulating the cholesterol biosynthesis pathway.

The invention also relates to the use of a combination of AZD9574 and an agent capable of modulating the cholesterol biosynthesis pathway in the manufacture of a medicament for the treatment of cancer, wherein the patient to be treated for cancer is already receiving treatment with an agent capable of modulating the cholesterol biosynthesis pathway for the treatment of a non-cancer related indication.

In an embodiment, the invention relates to a combination comprising AZD9574 and an agent capable of modulating the cholesterol biosynthesis pathway, for use in the treatment of cancer, wherein the patient to be treated for cancer is receiving treatment for cancer with AZD9574 and is subsequently prescribed an agent capable of modulating the cholesterol biosynthesis pathway.

The invention also relates to a method of treating cancer in a patient in need thereof, the method comprising administering to the patient a therapeutically effective amount of a poly (ADP-ribose) polymerase inhibitor (PARP inhibitor), wherein the patient is receiving treatment for hypercholesterolemia, hypertriglyceridemia or mixed hyperlipidemia. In particular the patient is receiving treatment for hypercholesterolemia, hypertriglyceridemia or mixed hyperlipidemia with a lipophilic statin. Alternatively, the patient may be receiving treatment for any other indication for which statins are used. For example, atherosclerosis and cardiovascular diseases (including coronary heart disease, angina, heart attacks, stroke).

Also disclosed is a method of treating cancer in a patient in need thereof, the method comprising administering to the patient a therapeutically effective amount of niraparib, wherein the patient is receiving treatment for hypercholesterolemia, hypertriglyceridemia or mixed hyperlipidemia. In particular the patient is receiving treatment for hypercholesterolemia, hypertriglyceridemia or mixed hyperlipidemia with a lipophilic statin. Alternatively, the patient may be receiving treatment for any other indication for which statins are used. For example, atherosclerosis and cardiovascular diseases (including coronary heart disease, angina, heart attacks, stroke).

In an embodiment, the patient may not be already receiving statin treatment, i.e. when the patient presents with cancer they may not already be receiving a therapeutically effective amount of a lipophilic statin but may benefit from such treatment in addition to a PARP inhibitor for the treatment of cancer. In some embodiments, the patient who may benefit from treatment with a lipophilic statin may be tested to determine if they have an indication for which statins are useful, e.g. hypercholesterolemia, hypertriglyceridemia or mixed hyperlipidemia, atherosclerosis and cardiovascular diseases (including coronary heart disease, angina, heart attacks, stroke).

In an embodiment, the patient may not be already receiving statin treatment when they present with cancer, but may benefit from treatment with a lipophilic statin in combination with a PARP inhibitor for the treatment of cancer.

In an embodiment, the patient may not be already receiving statin treatment, i.e. when the patient presents with cancer they may not already be receiving a therapeutically effective amount of a lipophilic statin but may benefit from such treatment in addition to niraparib for the treatment of cancer. In some embodiments, the patient who may benefit from treatment with a lipophilic statin may be tested to determine if they have an indication for which statins are useful, e.g. hypercholesterolemia, hypertriglyceridemia or mixed hyperlipidemia, atherosclerosis and cardiovascular diseases (including coronary heart disease, angina, heart attacks, stroke).

In an embodiment, the patient may not be already receiving statin treatment when they present with cancer, but may benefit from treatment with a lipophilic statin in combination with niraparib for the treatment of cancer. In other words, the patient does not have an indication for which treatment with a statin is useful.

In an embodiment, the patient may not be already receiving treatment with an agent capable of modulating the cholesterol biosynthesis pathway when they present with cancer, but may benefit from treatment with an agent capable of modulating the cholesterol biosynthesis pathway with niraparib for the treatment of cancer.

In another embodiment the method comprises administering to a cancer patient a therapeutically effective amount of a poly (ADP-ribose) polymerase inhibitor (PARP inhibitor) and a lipophilic statin, to prolong mPFS (mean progression free survival).

In other words, also contemplated is a method of treating cancer in a patient in need thereof, the method comprising administering to the patient a therapeutically effective amount of a PARP inhibitor and a lipophilic statin, wherein the patient has an mPFS following treatment which is longer relative to treatment with a PARP inhibitor alone.

Also contemplated is a method of treating cancer in a patient in need thereof, the method comprising administering to the patient a therapeutically effective amount of niraparib and a lipophilic statin, wherein the patient has an mPFS following treatment which is longer relative to treatment with niraparib alone. In an embodiment, the PARP inhibitor is not olaparib when the statin is simvastatin and the cancer to be treated is prostate cancer. In another embodiment, the PARP inhibitor is not olaparib when the lipophilic statin is atorvastatin and the cancer to be treated is breast cancer.

In some embodiments, the statin for use in the invention may be a CYP3A inhibitor, such as lovastatin or simvastatin. In such embodiments, it may be necessary to alter the dose of the PARP inhibitor accordingly, for example by lowering the dose. For example, it is described on the product label for olaparib that coadministration of olaparib with a strong or moderate CYP3A inhibitor should be avoided, however if a strong or moderate CYP3A inhibitor must be coadministered then the dose of olaparib should be reduced accordingly. In particular, if a strong CYP3A inhibitor is to be used in combination with olaparib then the recommended dose reduction is to 150 mg taken twice daily (equivalent to a daily dose of 300 mg). If a moderate CYP3A inhibitor is to be used in combination with olaparib then the recommended dose reduction is to 200 mg taken twice daily (equivalent to a daily dose of 400 mg).

In an embodiment, when the PARP inhibitor is olaparib, the lipophilic statin is selected from fluvastatin, lovastatin, pitavastatin and cerivastatin.

Therefore, the invention includes a method of treating cancer in a patient in need thereof, the method comprising administering to the patient (i) a therapeutically effective amount of a olaparib; and (ii) a therapeutically effective amount of a lipophilic statin selected from the group consisting of fluvastatin, lovastatin, pitavastatin and cerivastatin.

The invention also includes a method of treating prostate cancer in a patient in need thereof, the method comprising administering to the patient (i) a therapeutically effective amount of a olaparib; and (ii) a therapeutically effective amount of a lipophilic statin selected from the group consisting of atorvastatin, fluvastatin, lovastatin, pitavastatin and cerivastatin.

The invention also includes a method of treating breast cancer in a patient in need thereof, the method comprising administering to the patient (i) a therapeutically effective amount of a olaparib; and (ii) a therapeutically effective amount of a lipophilic statin selected from the group consisting of simvastatin, fluvastatin, lovastatin, pitavastatin and cerivastatin.

The invention also relates to a method of treating cancer in a patient in need thereof, the method comprising administering to the patient a therapeutically effective amount of a olaparib, wherein the patient is receiving a therapeutically effective amount of a lipophilic statin selected from the group consisting of fluvastatin, lovastatin, pitavastatin and cerivastatin.

The invention also relates to a method of treating prostate cancer in a patient in need thereof, the method comprising administering to the patient a therapeutically effective amount of a olaparib, wherein the patient is receiving a therapeutically effective amount of a lipophilic statin selected from the group consisting of atorvastatin, fluvastatin, lovastatin, pitavastatin and cerivastatin.

The invention also relates to a method of treating breast cancer in a patient in need thereof, the method comprising administering to the patient a therapeutically effective amount of a olaparib, wherein the patient is receiving a therapeutically effective amount of a lipophilic statin selected from the group consisting of simvastatin, fluvastatin, lovastatin, pitavastatin and cerivastatin.

In an embodiment, the PARP inhibitor is not olaparib.

In another embodiment the method comprises administering to a cancer patient a therapeutically effective amount of a poly (ADP-ribose) polymerase inhibitor (PARP inhibitor) and an agent that is capable of modulating the cholesterol biosynthesis pathway, such as an LSS inhibitor, to prolong mPFS (mean progression free survival).

In other words, in an embodiment is a method of treating cancer in a patient in need thereof, the method comprising administering to the patient a therapeutically effective amount of a PARP inhibitor and an agent that is capable of modulating the cholesterol biosynthesis pathway, such as an LSS inhibitor, wherein the patient has an mPFS following treatment which is longer relative to treatment with a PARP inhibitor alone.

In another embodiment is a method of treating cancer in a patient in need thereof, the method comprising administering to the patient a therapeutically effective amount of niraparib and an agent that is capable of modulating the cholesterol biosynthesis pathway, such as an LSS inhibitor, wherein the patient has an mPFS following treatment which is longer relative to treatment with niraparib alone.

Cancers that may be treated include: adenocarcinoma, basal cell carcinoma, squamous cell carcinoma, adenosquamous carcinoma, carcinosarcoma, melanoma, adrenal gland cancer, adrenocortical carcinoma, pheochromocytoma, breast cancer, ductal carcinoma in situ, lobular carcinoma, inflammatory breast cancer, invasive ductal carcinoma, Paget disease of the nipple, papillary breast cancer, medullary carcinoma, mammary carcinoma, anal cancer, cloacogenic carcinoma, anorectal melanoma, appendiceal cancer, appendiceal neuroendocrine tumour, appendiceal mucinous cystadenocarcinomas, colonic-type adenocarcinoma, signet-ring cell adenocarcinoma, goblet cell carcinomas/adenoneuroendocrine carcinomas, bile duct cancer, intrahepatic cholangiocarcinoma, extrahepatic cholangiocarcinoma, perihilar cholangiocarcinoma, distal extrahepatic cholangiocarcinoma, colorectal cancer, mucinous adenocarcinoma, gastrointestinal carcinoid tumours, gastrointestinal stromal tumours, primary colorectal lymphomas, leiomyosarcoma, esophageal cancer, small cell carcinoma, leiomyoma, gallbladder cancer, non-papillary adenocarcinoma, papillary adenocarcinoma, stomach cancer, gastric adenocarcinoma, liver cancer, hepatocellular carcinoma, fibrolamellar carcinoma, angiosarcoma, lymphangiosarcoma, hemangiosarcoma, hepatoblastoma, pancreatic cancer, ductal adenocarcinoma, acinar adenocarcinoma, acinar cell carcinoma, colloid carcinoma, giant cell tumour, hepatoid carcinoma, mucinous cystic neoplasms, pancreatoblastoma, serous cystadenoma, intraductal papillary mucinous neoplasm, pancreatic neuroendocrine tumour, gastrinoma, insulinoma, glucagonoma, VIPoma, somatostatinoma, PPoma, small intestine cancer, eye cancer, intraocular melanoma, intraocular lymphoma, intraocular retinoblastoma, conjunctival melanoma, eyelid carcinoma, sebaceous carcinoma, lacrimal gland tumour, malignant mixed epithelial tumour, adenoid cystic carcinoma, bladder cancer, urothelial carcinoma, kidney cancer, renal cell carcinoma (RCC), clear cell RCC, papillary RCC, chromophobe RCC, collecting duct RCC, multilocular cystic RCC, renal mucinous tubular and spindle cell carcinoma, tubulocystic RCC, thyroid-like follicular RCC, acquired cystic kidney disease-associated RCC, RCC with t(6;11) translocation (TFEB), hybrid oncocytoma/chromophobe RCC, Wilms tumor, penile cancer, prostate cancer, castration-resistant prostate cancer, transitional cell carcinoma, testicular cancer, seminoma, classical seminoma, spermatocytic seminoma, non-seminoma, embryonal carcinoma, yolk sac carcinoma, choriocarcinoma, teratoma, Leydig cell tumours, Sertoli cell tumours, carcinoma of the rete testis, urethral cancer, extracranial germ cell tumour, germinoma, gonadoblastoma, mixed germ cell tumour, extragonadal germ cell tumour, endodermal sinus tumours, cervical cancer, endometrial cancer, ovarian cancer, fallopian tube cancer, epithelial carcinoma, dysgerminoma, sex cord stromal tumours, gestational trophoblastic tumour, primary peritoneal cancer, uterine sarcoma, uterine papillary serous carcinoma, vaginal cancer, clear cell adenocarcinoma, vulvar cancer, verrucous carcinoma, head and neck cancer, head and neck squamous cell carcinoma, pharyngeal cancer, hypopharyngeal cancer, nasopharyngeal cancer, oropharyngeal cancer, non-keratinising squamous cell carcinoma, undifferentiated carcinoma, laryngeal cancer, oral cavity cancer, mouth cancer, mucoepidermoid carcinoma, paranasal sinus and nasal cavity cancer, esthesioneuroblastoma, salivary gland cancer, epithelial-myoepithelial carcinoma, parathyroid cancer, thyroid cancer, papillary thyroid carcinoma, follicular thyroid carcinoma, Hurthle cell carcinoma, medullary thyroid carcinoma, anaplastic thyroid carcinoma, paraganglioma, carotid paraganglioma, jugulotympanic paraganglioma, vagal paraganglioma, leukemia, acute lymphoblastic leukemia, T-lymphoblastic leukemia, precursor B-cell lymphoblastic leukemia, acute myelogenous leukemia, acute promyelocytic leukemia, acute myelomonocytic leukemia, acute monocytic leukemia, acute megakaryoblastic leukemia, erythroleukemia, chronic lymphocytic leukemia, B-cell chronic lymphocytic leukemia, B-cell prolymphocytic leukemia, T-cell prolymphocytic leukemia, large granular lymphocytic leukemia, T-cell large granular lymphocytic leukemia, NK-cell granular lymphocytic leukemia, hairy cell leukemia, chronic myelogenous leukemia, chronic myelomonocytic leukemia, chronic neutrophilic leukemia, chronic eosinophilic leukemia, plasma cell leukemia, lymphoma, Hodgkin's lymphoma, classical Hodgkin's lymphoma, nodular sclerosing classical Hodgkin's lymphoma, mixed cellularity classical Hodgkin's lymphoma, lymphocyte-rich classical Hodgkin's lymphoma, lymphocyte-depleted classical Hodgkin's lymphoma, nodular lymphocyte-predominant Hodgkin's lymphoma, non-Hodgkin's lymphoma, diffuse large B-cell lymphoma, primary mediastinal B-cell lymphoma, primary effusion lymphoma, T-cell/histiocyte-rich large B-cell lymphoma, lymphoplasmacytic lymphoma, lymphoblastic lymphoma, small lymphocytic lymphoma, double hit/triple hit lymphoma, Burkitt lymphoma, Burkitt-like lymphoma, small non-cleaved cell lymphoma, follicular lymphoma, follicular large-cell lymphoma, immunoblastic lymphoma, intravascular large-cell lymphoma, primary splenic lymphoma, anaplastic large-cell lymphoma, mantle cell lymphoma, marginal zone lymphoma (MZL), extranodal MZL, nodal MZL, splenic MZL, splenic MZL with villous lymphocytes, peripheral T-cell lymphoma, angioimmunoblastic T-cell lymphoma, adult T-cell lymphoma/leukemia, extranodal NK/T-cell lymphoma, enteropathy-associated T-cell lymphoma, hepatosplenic T-cell lymphoma, subcutaneous panniculitis-like T-cell lymphoma, T-cell non-Hodgkin's lymphoma not otherwise specified, gamma/delta T-cell lymphoma, mucosa-associated-lymphoid tissue lymphoma, post-transplant lymphoproliferative disorder, HIV-associated lymphoma, Langerhans cell histiocytosis, multiple myeloma, smoldering multiple myeloma, active multiple myeloma, plasmacytoma, solitary plasmacytoma of bone, extramedullary plasmacytoma, primary amyloidosis, myelodysplastic syndromes, refractory anaemia, refractory anaemia with ring sideroblasts, refractory anaemia with excess blasts, refractory anaemia with excess blasts in transformation, myeloproliferative neoplasms, polycythemia vera, essential thrombocythemia, myelofibrosis, systemic mastocytosis, bone cancer, Ewing sarcoma, osteosarcoma, intramedullary osteosarcoma, juxtacortical osteosarcoma, extraskeletal osteosarcoma, malignant fibrous histiocytoma of bone, chordoma, classic chordoma, chondroid chordoma, dedifferentiated chordoma, chondrosarcoma, conventional chondrosarcoma, clear cell chondrosarcoma, myxoid chondrosarcoma, mesenchymal chondrosarcoma, dedifferentiated chondrosarcoma, rhabdomyosarcoma, embryonal rhabdomyosarcoma, alveolar rhabdomyosarcoma, botryoid rhabdomyosarcoma, pleomorphic rhabdomyosarcoma, soft tissue sarcoma, extraosseus sarcoma, dermatofibrosarcoma protuberans, epithelioid sarcoma, Kaposi's sarcoma, liposarcoma, malignant peripheral nerve sheath tumour, fibrosarcoma, myxosarcoma, synovioma, brain cancer, anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical teratoid/rhabdoid tumour, pinealoma, neuroblastoma, primary CNS lymphoma, primitive neuroectodermal tumour, diffuse intrinsic pontine glioma, lung cancer, non-small cell lung cancer (NSCLC), NSCLC undifferentiated, small cell lung cancer, pleuropulmonary blastoma, bronchogenic carcinoma, malignant mesothelioma, malignant pleural mesothelioma, malignant peritoneal mesothelioma, thymoma, thymic carcinoma, skin cancer, keratoacanthoma, sebaceous gland carcinoma, sweat gland adenocarcinoma, apocrine carcinoma, eccrine carcinoma, clear cell eccrine carcinoma, Merkel cell carcinoma, cutaneous T cell lymphoma, mycosis fungoides, Sezary syndrome, chondroid syringoma, HPV-associated cancers, tumours containing transformed cells, tumours containing cells in precancerous states, precancerous hyperplasia, precancerous metaplasia, precancerous dysplasia, carcinoma in situ, mixed tumour, malignant mixed tumour, and complex carcinoma.

In an embodiment, the cancer to be treated is a solid tumour. Solid tumours may be benign or malignant. Different types of tumours are named for the type of cells that form them. Examples of solid tumours are sarcomas, carcinomas and lymphomas.

Cancers that are considered to be solid tumours include, but are not limited to: adenocarcinoma, basal cell carcinoma, squamous cell carcinoma, adenosquamous carcinoma, carcinosarcoma, melanoma, adrenal gland cancer, adrenocortical carcinoma, pheochromocytoma, breast cancer, ductal carcinoma in situ, lobular carcinoma, inflammatory breast cancer, invasive ductal carcinoma, Paget disease of the nipple, papillary breast cancer, medullary carcinoma, mammary carcinoma, anal cancer, cloacogenic carcinoma, anorectal melanoma, appendiceal cancer, appendiceal neuroendocrine tumour, appendiceal mucinous cystadenocarcinomas, colonic-type adenocarcinoma, signet-ring cell adenocarcinoma, goblet cell carcinomas/adenoneuroendocrine carcinomas, bile duct cancer, intrahepatic cholangiocarcinoma, extrahepatic cholangiocarcinoma, perihilar cholangiocarcinoma, distal extrahepatic cholangiocarcinoma, colorectal cancer, mucinous adenocarcinoma, gastrointestinal carcinoid tumours, gastrointestinal stromal tumours, primary colorectal lymphomas, leiomyosarcoma, esophageal cancer, small cell carcinoma, leiomyoma, gallbladder cancer, non-papillary adenocarcinoma, papillary adenocarcinoma, stomach cancer, gastric adenocarcinoma, liver cancer, hepatocellular carcinoma, fibrolamellar carcinoma, angiosarcoma, lymphangiosarcoma, hemangiosarcoma, hepatoblastoma, pancreatic cancer, ductal adenocarcinoma, acinar adenocarcinoma, acinar cell carcinoma, colloid carcinoma, giant cell tumour, hepatoid carcinoma, mucinous cystic neoplasms, pancreatoblastoma, serous cystadenoma, intraductal papillary mucinous neoplasm, pancreatic neuroendocrine tumour, gastrinoma, insulinoma, glucagonoma, VIPoma, somatostatinoma, PPoma, small intestine cancer, eye cancer, intraocular melanoma, intraocular lymphoma, intraocular retinoblastoma, conjunctival melanoma, eyelid carcinoma, sebaceous carcinoma, lacrimal gland tumour, malignant mixed epithelial tumour, adenoid cystic carcinoma, bladder cancer, urothelial carcinoma, kidney cancer, renal cell carcinoma (RCC), clear cell RCC, papillary RCC, chromophobe RCC, collecting duct RCC, multilocular cystic RCC, renal mucinous tubular and spindle cell carcinoma, tubulocystic RCC, thyroid-like follicular RCC, acquired cystic kidney disease-associated RCC, RCC with t (6;11) translocation (TFEB), hybrid oncocytoma/chromophobe RCC, Wilms tumor, penile cancer, prostate cancer, castration-resistant prostate cancer, transitional cell carcinoma, testicular cancer, seminoma, classical seminoma, spermatocytic seminoma, non-seminoma, embryonal carcinoma, yolk sac carcinoma, choriocarcinoma, teratoma, Leydig cell tumours, Sertoli cell tumours, carcinoma of the rete testis, urethral cancer, extracranial germ cell tumour, germinoma, gonadoblastoma, mixed germ cell tumour, extragonadal germ cell tumour, endodermal sinus tumours, cervical cancer, endometrial cancer, ovarian cancer, fallopian tube cancer, epithelial carcinoma, dysgerminoma, sex cord stromal tumours, gestational trophoblastic tumour, primary peritoneal cancer, uterine sarcoma, uterine papillary serous carcinoma, vaginal cancer, clear cell adenocarcinoma, vulvar cancer, verrucous carcinoma, head and neck cancer, head and neck squamous cell carcinoma, pharyngeal cancer, hypopharyngeal cancer, nasopharyngeal cancer, oropharyngeal cancer, non-keratinising squamous cell carcinoma, undifferentiated carcinoma, laryngeal cancer, oral cavity cancer, mouth cancer, mucoepidermoid carcinoma, paranasal sinus and nasal cavity cancer, esthesioneuroblastoma, salivary gland cancer, epithelial-myoepithelial carcinoma, parathyroid cancer, thyroid cancer, papillary thyroid carcinoma, follicular thyroid carcinoma, Hurthle cell carcinoma, medullary thyroid carcinoma, anaplastic thyroid carcinoma, paraganglioma, carotid paraganglioma, jugulotympanic paraganglioma, vagal paraganglioma, lymphoma, Hodgkin's lymphoma, classical Hodgkin's lymphoma, nodular sclerosing classical Hodgkin's lymphoma, mixed cellularity classical Hodgkin's lymphoma, lymphocyte-rich classical Hodgkin's lymphoma, lymphocyte-depleted classical Hodgkin's lymphoma, nodular lymphocyte-predominant Hodgkin's lymphoma, non-Hodgkin's lymphoma, diffuse large B-cell lymphoma, primary mediastinal B-cell lymphoma, primary effusion lymphoma, T-cell/histiocyte-rich large B-cell lymphoma, lymphoplasmacytic lymphoma, lymphoblastic lymphoma, small lymphocytic lymphoma, double hit/triple hit lymphoma, Burkitt lymphoma, Burkitt-like lymphoma, small non-cleaved cell lymphoma, follicular lymphoma, follicular large-cell lymphoma, immunoblastic lymphoma, intravascular large-cell lymphoma, primary splenic lymphoma, anaplastic large-cell lymphoma, mantle cell lymphoma, marginal zone lymphoma (MZL), extranodal MZL, nodal MZL, splenic MZL, splenic MZL with villous lymphocytes, peripheral T-cell lymphoma, angioimmunoblastic T-cell lymphoma, adult T-cell lymphoma/leukemia, extranodal NK/T-cell lymphoma, enteropathy-associated T-cell lymphoma, hepatosplenic T-cell lymphoma, subcutaneous panniculitis-like T-cell lymphoma, T-cell non-Hodgkin's lymphoma not otherwise specified, gamma/delta T-cell lymphoma, mucosa-associated-lymphoid tissue lymphoma, post-transplant lymphoproliferative disorder, HIV-associated lymphoma, plasmacytoma, solitary plasmacytoma of bone, extramedullary plasmacytoma, systemic mastocytosis, bone cancer, Ewing sarcoma, osteosarcoma, intramedullary osteosarcoma, juxtacortical osteosarcoma, extraskeletal osteosarcoma, malignant fibrous histiocytoma of bone, chordoma, classic chordoma, chondroid chordoma, dedifferentiated chordoma, chondrosarcoma, conventional chondrosarcoma, clear cell chondrosarcoma, myxoid chondrosarcoma, mesenchymal chondrosarcoma, dedifferentiated chondrosarcoma, rhabdomyosarcoma, embryonal rhabdomyosarcoma, alveolar rhabdomyosarcoma, botryoid rhabdomyosarcoma, pleomorphic rhabdomyosarcoma, soft tissue sarcoma, extraosseus sarcoma, dermatofibrosarcoma protuberans, epithelioid sarcoma, Kaposi's sarcoma, liposarcoma, malignant peripheral nerve sheath tumour, fibrosarcoma, myxosarcoma, synovioma, brain cancer, anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical teratoid/rhabdoid tumour, pinealoma, neuroblastoma, primary CNS lymphoma, primitive neuroectodermal tumour, diffuse intrinsic pontine glioma, lung cancer, non-small cell lung cancer (NSCLC), NSCLC undifferentiated, small cell lung cancer, pleuropulmonary blastoma, bronchogenic carcinoma, malignant mesothelioma, malignant pleural mesothelioma, malignant peritoneal mesothelioma, thymoma, thymic carcinoma, skin cancer, keratoacanthoma, sebaceous gland carcinoma, sweat gland adenocarcinoma, apocrine carcinoma, eccrine carcinoma, clear cell eccrine carcinoma, Merkel cell carcinoma, cutaneous T cell lymphoma, mycosis fungoides, Sezary syndrome, chondroid syringoma, HPV-associated cancers, tumours containing transformed cells, tumours containing cells in precancerous states, precancerous hyperplasia, precancerous metaplasia, precancerous dysplasia, carcinoma in situ, mixed tumour, malignant mixed tumour, and complex carcinoma.

In some embodiments, the cancer may be a haematological cancer, such as leukaemia, lymphoma or multiple myeloma.

In an embodiment, the cancer to be treated is characterised by being dependent on the steroid biosynthesis pathway. In an embodiment, the cancer to be treated may be selected from the group consisting of breast, colorectal, gastric, head & neck, kidney, liver, lung, ovarian, pancreatic, skin, sarcoma, brain, endometrial, thyroid, neuroendocrine, bladder, cervical, melanoma and prostate cancer.

In an embodiment, the cancer to be treated is selected from the group consisting of breast, colorectal, gastric, head & neck, kidney, liver, lung, ovarian, pancreatic and skin cancer.

In an embodiment, the cancer to be treated is selected from the group consisting of breast, colorectal, gastric, head & neck, liver, lung, ovarian, pancreatic and skin cancer.

In a particular embodiment, the cancer to be treated is selected from the group consisting of breast, ovarian and lung cancer.

In one embodiment, the cancer is breast cancer. In particular, triple-negative breast cancer (TNBC) or human epidermal growth factor 2 negative (HER2−) BRCA-mutated breast cancer.

In one embodiment, the cancer is lung cancer. In particular, non-small cell lung cancer.

In one embodiment, the cancer is ovarian cancer.

In an embodiment, the cancer is cervical cancer.

In an embodiment, the cancer is head & neck cancer.

In an embodiment, when the PARP inhibitor is niraparib, more specifically niraparib tosylate monohydrate, the cancer is selected from ovarian cancer, breast cancer and lung cancer, particularly ovarian cancer.

Breast cancer includes, but is not limited to those including ductal carcinoma in situ, lobular carcinoma, inflammatory breast cancer, invasive ductal carcinoma, Paget disease of the nipple, papillary breast cancer, medullary carcinoma, mammary carcinoma, PAM-50 classes including basal (triple-negative), HER2 positive, luminal A, luminal B and normal-like) and breast cancer in patients having a mutation in BRCA1 and/or BRCA2.

Colorectal cancer includes, but is not limited to those including adenocarcinoma (including mucinous adenocarcinoma and signet ring cell adenocarcinoma), gastrointestinal carcinoid tumours, gastrointestinal stromal tumours (GISTs), primary colorectal lymphomas, leiomyosarcoma and melanoma. Head and neck cancer includes, but is not limited to those including head and neck squamous cell carcinoma; pharyngeal (hypopharyngeal, nasopharyngeal, oropharyngeal) including squamous cell carcinoma, non-keratinising squamous cell carcinoma and undifferentiated carcinoma; laryngeal including squamous cell carcinoma, sarcoma, adenocarcinoma, plasmacytomal; oral cavity including squamous cell carcinoma, sarcoma, adenoid cystic carcinoma, mucoepidermoid carcinoma, adenocarcinoma and melanoma; paranasal sinus and nasal cavity including squamous cell carcinoma, adenocarcinoma, melanoma, esthesioneuroblastoma, lymphoma and sarcoma; salivary gland cancer including mucoepidermoid carcinoma, adenoid cystic carcinoma, adenocarcinoma, carcinosarcoma, squamous cell carcinoma and epithelial-myoepithelial carcinoma; parathyroid; thyroid including papillary thyroid cancer, follicular thyroid cancer, hurthle cell cancer, medullary thyroid cancer and anaplastic thyroid cancer; paraganglioma (HNPGL) including carotid paraganglioma, jugulotympanic paraganglioma and vagal paraganglioma.

Kidney cancer includes, but is not limited to those including renal cell carcinoma (RCC) including clear cell RCC, papillary RCC, chromophobe RCC, collecting duct RCC, multiocular cystic RCC, medullary carcinoma, renal mucinous tubular and spindle cell carcinoma, tubulocystic RCC, thyroid-like follicular RCC, acquired cystic kidney disease associated RCC, RCC with t (6;11) translocation (TFEB) and hybrid oncocytoma/chromophobe RCC; urothelial carcinoma; sarcoma; Wilms tumour; and lymphoma.

Liver cancer includes, but is not limited to those including hepatocellular carcinoma (HCC) including fibrolamellaer carcinoma; angiosarcoma; and hepatoblastoma.

Lung cancer includes, but is not limited to those including non-small cell lung cancer (NSCLC) including adenocarcinoma, squamous cell carcinoma, large cell carcinoma and NSCLC undifferentiated; small cell lung cancer; pleuropulmonary blastoma; bronchogenic carcinoma; tracheobronchial including squamous cell carcinoma, adenoid cystic carcinoma and carcinoid tumours; and malignant mesothelioma including malignant pleural mesothelioma and malignant peritoneal mesothelioma.

Ovarian cancer includes, but is not limited to those including epithelial carcinoma; dysgerminoma, teratoma; endodermal sinus tumours; embryonal carcinoma; sex cord stromal tumours; and ovarian cancer in patients having a mutation in BRCA1 and/or BRCA2.

Pancreatic cancer includes, but is not limited to those including exocrine including adenocarcinoma (ductal adenocarcinoma and acinar adenocarcinoma), acinar cell carcinoma, squamous cell carcinoma, adenosquamous carcinoma, colloid carcinoma, giant cell tumour, hepatoid carcinoma, mucinous cystic neoplasms, pancreatoblastoma, serous cystadenoma, signet-ring cell carcinoma and intraductal papillary mucinous neoplasm (IPMN); and pancreatic neuroendocrine tumours (PNETs) including gastrinomas, insulinomas, glucagonoma, VIPomas, somatostatinomas and PPomas.

Skin cancer includes, but is not limited to those including melanoma, keratoacanthoma, basal cell carcinoma, squamous cell carcinoma, sebaceous gland carcinoma, sweat gland adenocarcinoma, apocrine carcinoma, eccrine carcinoma, clear cell eccrine carcinoma, merkel cell carcinoma, chondroid syringoma and cutaneous T cell lymphoma (including mycosis fungoides).

Brain cancer includes, but is not limited to those including anaplastic astrocytoma, glioblastoma, glioblastoma multiforme, meningioma, pituitary carcinoma, schwannoma, oligodendroglioma, ependymoma, medulloblastoma, astrocytoma, brainstem glioma, atypical Teratoid/Rhabdoid tumour, pinealoma and diffuse intrinsic pontine glioma.

Endometrial cancer includes, but is not limited to those including adenocarcinoma and sarcoma.

Thyroid cancer includes, but is not limited to those including parathyroid; papillary thyroid cancer, follicular thyroid cancer, hurthle cell cancer, medullary thyroid cancer and anaplastic thyroid cancer.

Bladder cancer includes, but is not limited to those including urothelial carcinoma, squamous cell carcinoma, adenocarcinoma, sarcoma and small cell carcinoma.

Cervical cancer includes, but is not limited to those including squamous cell carcinoma, adenocarcinoma, adenosquamous carcinoma and small cell carcinoma.

Prostate cancer includes, but is not limited to those including adenocarcinoma including acinar adenocarcinoma and ductal adenocarcinoma; transitional cell carcinoma; squamous cell carcinoma; small cell carcinoma; large cell carcinoma; and sarcoma including leiomyosarcoma and rhabdomyosarcoma.

In an embodiment, the definition of cancer is also intended to include stable disease. Stable disease may be after treatment with platinum therapy.

The combination according to the present invention and methods of treating cancer with the combination of a PARP inhibitor and lipophilic statin may be used in the treatment of metastatic tumours.

The combination according to the present invention and methods of treating cancer with the combination of a PARP inhibitor and lipophilic statin may be particularly useful in in the treatment of metastatic tumours located in the brain.

In an embodiment, there is provided a method of treating brain cancer (primary or secondary) in a patient in need thereof, the method comprising administering to the patient a therapeutically effective amount of a PARP inhibitor and an agent capable of modulating the cholesterol biosynthesis pathway. In an embodiment, the agent capable of modulating the cholesterol biosynthesis pathway is an LSS inhibitor. In an embodiment, the agent capable of modulating the cholesterol biosynthesis pathway is a lipophilic statin.

In an embodiment, there is provided a method of treating brain cancer (primary or secondary) in a patient in need thereof, the method comprising administering to the patient a therapeutically effective amount of AZD9574 and an agent capable of modulating the cholesterol biosynthesis pathway. In an embodiment, the agent capable of modulating the cholesterol biosynthesis pathway is an LSS inhibitor. In an embodiment, the agent capable of modulating the cholesterol biosynthesis pathway is a lipophilic statin.

The combination according to the present invention and methods of treating cancer with the combination of a PARP inhibitor and lipophilic statin may be used in the treatment of cancer which is deficient in Homologous Recombination (HR) dependent DNA DSB repair activity. In other words, the combination may be useful in the treatment of a patient having a cancer associated with a homologous recombination deficiency positive status.

The HR dependent DNA DSB repair pathway repairs double-strand breaks (DSBs) in DNA via homologous mechanisms to reform a continuous DNA helix (Nat. Genet. (2001) 27 (3): 247-254). The components of the HR dependent DNA DSB repair pathway include, but are not limited to, ATM (NM-000051), RAD51 (NM-002875), RAD51LI (NM-002877), RAD51C (NM-002876), RAD51L3 (NM-002878), DMCI (NM-007068), XRCC2 (NM7005431), XRCC3 (NM-005432), RAD52 (NM-002879), RAD54L (NM-003579), RAD54B (NM-012415), BRCA1 (NM-007295), BRCA2 (NM-000059), RAD5O (NM-005732), MRE11A (NM-005590), NBSI (NM-002485), ADPRT (PARP-1), ADPRTL2, (PARP2) CTPS, RPA, RPAI, RPA2, RPA3, XPD, ERCCI, XPF, MMS19, RAD51p, RAD51D, DMC1, XRCCR, XRCC3, RAD54, NB51, WRN, BLMKU70, RU8O, ATR, CHKI, CHK2, FANCA, FANCB, FANCC, FANCD2, FANCE, FANCF, FANCG, RAD1, RAD9, BARD1 (NM_000465), PALB2 (NM_024675), BLM (NM_000057) and BRIP1 (NM_032043). Other proteins involved in the HR dependent DNA DSB repair pathway include regulatory factors such as EMSY (Cell (2003) 115:523-535).

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells. Deficiencies in HR dependent DNA DSB repair may arise from a mutation that leads to under-expression or lack of expression of any component in the pathway, including, but not limited to those defined above.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterized in the art (see for example, Science (2001) 291:1284-1289) and include the components listed above.

In an embodiment, the cancer cells have a BRCA1 and/or BRCA2 deficient phenotype. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Cell (2003) 115:523-535).

BRCA1 and BRCA2 are known tumour suppressors whose wild-type alleles are frequently lost in tumors of heterozygous carriers (Oncogene, (2002) 21 (58): 8981-93; Trends Mal Med., (2002) 8 (12): 571-6). The association of BRCA-1 and/or BRCA-2 mutations with breast cancer has been well-characterized (Exp Clin Cancer Res., (2002) 21 (3 Suppl): 9-12). The association with ovarian cancer is equally well-characterised. Amplification of the EMSY gene, which encodes a BRCA-2 binding factor, is also known to be associated with breast and ovarian cancer. Carriers of mutations in BRCA1 and/or BRCA2 are also at elevated risk of cancer of the ovary, prostate and pancreas. In addition, carriers of mutations in BRCA1 and/or BRCA3 are also at elevated risk of melanoma and male breast cancer (Mersch et al., Cancer (2015) 121 (2): 269-275). The detection of variation in BRCA1 and BRCA2 is well-known in the art and is described, for example in EP 699 754, EP 705 903, Genet. Test (1992) 1:75-83; Cancer Treat Res (2002) 107:29-59; Neoplasm (2003) 50 (4): 246-50; Ceska Gynekol (2003) 68 (1): 11-16). Determination of amplification of the BRCA2 binding factor EMSY is described in Cell 115:523-535. PARP inhibitors have been demonstrated as being useful for the specific killing of BRCA1 and BRCA2 deficient tumours (Nature (2005) 434:913-916 and 917-920).

The treatment of cancers that exhibit HRD are included within the scope of the present invention. In particular, HRD is enriched in ovarian, prostate, pancreatic and breast cancer, where defects in BRCA1, BRCA2, RAD51, RAD51C, RAD51D or PALB2 are most prevalent. Therefore, the present invention is also useful in the treatment of such cancers. Patients having a cancer characterised in this way may be defined as having a HRD positive status or as being HR deficient.

For the avoidance of doubt, HRD is homologous recombination deficiency and a patient is characterised as being either HR proficient or HR deficient. HR proficient is also understood to mean that a patient is HRD negative and HR deficient is understood to mean that a patient is HRD positive.

When a patient is characterised for treatment with reference to their HRD status, it is preferable that they are selected based on an approved companion diagnostic.

For example, the molecular (sequence)-based in vitro diagnostic test available from Myriad Genetics, Inc., known as “MyChoice”. Alternatively using the methods described in WO 2020/225753, US 2020/255909 or WO 2021/070039, amongst others known in the art. Another example of a companion diagnostic is the FoundationOneCDx and BRACAnalysis CDx test, recently approved to identify patients with metastatic castration-resistant prostate cancer (mCRPC) with homologous recombination repair (HRR) mutations.

In an embodiment, the patient may be characterised as being HR proficient or HR deficient.

The present invention also provides a combination comprising a PARP inhibitor and a lipophilic statin for use in the treatment of HR deficient tumours or where the patient is characterised as HR deficient. In particular, in the treatment of ovarian, prostate, pancreatic or breast cancer associated with being HR deficient.

In another embodiment, the invention also provides a combination comprising a PARP inhibitor and a lipophilic statin for use in the treatment of BRCA1 or BRCA2 deficient tumors. In particular, in the treatment of ovarian, prostate, pancreatic or breast cancer associated with BRCA1 and/or BRCA2 deficient tumours.

The patient may have a cancer characterised by having a deleterious or suspected deleterious mutation in a component of the HR dependent DNA DSB repair pathway, including, but not limited to those defined above.

In an embodiment, the patient may have a cancer characterised by having a deleterious or suspected deleterious mutation in BRCA1 and/or BRCA2. In an embodiment, the cancer characterised by having a deleterious or suspected deleterious mutation in BRCA1 and/or BRCA2 is ovarian cancer or breast cancer. In particular, ovarian cancer.

In an embodiment, the PARP inhibitor is administered before a patient receives platinum-based therapy for the treatment of cancer.

In an embodiment, the PARP inhibitor and the lipophilic statin is administered before a patient receives platinum-based therapy for the treatment of cancer.

In an embodiment, the PARP inhibitor is administered as a maintenance therapy following a complete or partial response to a platinum-based therapy. In other words, the PARP inhibitor is administered following at least one cycle of platinum-based therapy. The lipophilic statin may also be administered in this regimen, such that a PARP inhibitor and a lipophilic statin are administered following a complete or partial response to a platinum-based therapy or following at least one cycle of platinum-based therapy.

In an embodiment, the PARP inhibitor is administered as a maintenance therapy in patients having recurrent cancer, in particular recurrent ovarian cancer, following a complete or partial response to platinum-based therapy. The lipophilic statin may also be administered in this regimen, such that a PARP inhibitor and a lipophilic statin are administered in patients having recurrent cancer, in particular recurrent ovarian cancer, following a complete or partial response to a platinum-based therapy.

In another embodiment, the PARP inhibitor is administered in patients, in particular patients having ovarian cancer, having had three or more prior chemotherapy treatments and whose cancer is associated with HRD positive status. The HRD positive status may be characterised by a deleterious or suspected deleterious BRCA mutation or in patients with genomic instability and who have progressed more than six months after response to the last platinum-based therapy. The lipophilic statin may also be administered in this regimen, such that the PARP inhibitor and the lipophilic statin is administered in patients, in particular patients having ovarian cancer, having had three or more prior chemotherapy treatments and whose cancer is associated with HRD positive status.

In an embodiment, administration of the PARP inhibitor and/or statin is administered until disease progression occurs.

In an embodiment, the PARP inhibitor and lipophilic statin is administered in patients characterised by having a cancer which is HR proficient.

In an embodiment, niraparib is administered before a patient receives platinum-based therapy for the treatment of cancer.

In an embodiment, niraparib and the lipophilic statin is administered before a patient receives platinum-based therapy for the treatment of cancer.

In another embodiment, niraparib is administered as a maintenance therapy following a complete or partial response to a platinum-based therapy. In other words, niraparib is administered following at least one cycle of platinum-based therapy. The lipophilic statin may also be administered in this regimen, such that a niraparib and a lipophilic statin are administered following a complete or partial response to a platinum-based therapy or following at least one cycle of platinum-based therapy.

In an embodiment, niraparib is administered as a maintenance therapy in patients having recurrent cancer, in particular recurrent ovarian cancer, following a complete or partial response to platinum-based therapy. The lipophilic statin may also be administered in this regimen, such that niraparib and a lipophilic statin are administered in patients having recurrent cancer, in particular recurrent ovarian cancer, following a complete or partial response to a platinum-based therapy.

In another embodiment, niraparib is administered in patients, in particular patients having ovarian cancer, having had three or more prior chemotherapy treatments and whose cancer is associated with HRD positive status. The HRD positive status may be characterised by a deleterious or suspected deleterious BRCA mutation or in patients with genomic instability and who have progressed more than six months after response to the last platinum-based therapy. The lipophilic statin may also be administered in this regimen, such that niraparib and the lipophilic statin is administered in patients, in particular patients having ovarian cancer, having had three or more prior chemotherapy treatments and whose cancer is associated with HRD positive status. In an embodiment, niraparib and lipophilic statin is administered in patients characterised by having a cancer which is HR proficient.

In an embodiment, olaparib is administered before a patient receives platinum-based therapy for the treatment of cancer.

In an embodiment, olaparib and the lipophilic statin is administered before a patient receives platinum-based therapy for the treatment of cancer.

In another embodiment, olaparib is administered as a maintenance therapy following a complete or partial response to a platinum-based therapy. In other words, olaparib is administered following at least one cycle of platinum-based therapy. The lipophilic statin may also be administered in this regimen, such that a olaparib and a lipophilic statin are administered following a complete or partial response to a platinum-based therapy or following at least one cycle of platinum-based therapy.

In an embodiment, olaparib is administered as a maintenance therapy in patients having recurrent cancer, in particular recurrent ovarian cancer, following a complete or partial response to platinum-based therapy. The lipophilic statin may also be administered in this regimen, such that olaparib and a lipophilic statin are administered in patients having recurrent cancer, in particular recurrent ovarian cancer, following a complete or partial response to a platinum-based therapy.

In another embodiment, olaparib is administered in patients, in particular patients having ovarian cancer, having had three or more prior chemotherapy treatments and whose cancer is HR deficient. In an embodiment, olaparib is administered in patients whose cancer is HR deficient in BRCA1 and/or BRCA2. The lipophilic statin may also be administered in this regimen, such that olaparib and a lipophilic statin are administered in patients, in particular patients having ovarian cancer, having had three or more prior chemotherapy treatments and whose cancer is HR deficient.

In an embodiment, olaparib and lipophilic statin is administered in patients characterised by having a cancer which is HR proficient.

In an embodiment, AZD5305 is administered before a patient receives platinum-based therapy for the treatment of cancer.

In an embodiment, AZD5305 and the lipophilic statin is administered before a patient receives platinum-based therapy for the treatment of cancer.

In another embodiment, AZD5305 is administered as a maintenance therapy following a complete or partial response to a platinum-based therapy. In other words, AZD5305 is administered following at least one cycle of platinum-based therapy. The lipophilic statin may also be administered in this regimen, such that AZD5305 and a lipophilic statin are administered following a complete or partial response to a platinum-based therapy or following at least one cycle of platinum-based therapy.

In an embodiment, AZD5305 is administered as a maintenance therapy in patients having recurrent cancer, in particular recurrent ovarian cancer, following a complete or partial response to platinum-based therapy. The lipophilic statin may also be administered in this regimen, such that AZD5305 and a lipophilic statin are administered in patients having recurrent cancer, in particular recurrent ovarian cancer, following a complete or partial response to a platinum-based therapy.

In another embodiment, AZD5305 is administered in patients, in particular patients having ovarian cancer, having had three or more prior chemotherapy treatments and whose cancer is HR deficient. In an embodiment, AZD5305 is administered in patients whose cancer is HR deficient in BRCA1 and/or BRCA2. The lipophilic statin may also be administered in this regimen, such that AZD5305 and a lipophilic statin are administered in patients, in particular patients having ovarian cancer, having had three or more prior chemotherapy treatments and whose cancer is HR deficient.

In an embodiment, AZD5305 and lipophilic statin is administered in patients characterised by having a cancer which is HR proficient.

In an embodiment, AZD9574 is administered before a patient receives platinum-based therapy for the treatment of cancer.

In an embodiment, AZD9574 and the lipophilic statin is administered before a patient receives platinum-based therapy for the treatment of cancer.

In another embodiment, AZD9574 is administered as a maintenance therapy following a complete or partial response to a platinum-based therapy. In other words, AZD9574 is administered following at least one cycle of platinum-based therapy. The lipophilic statin may also be administered in this regimen, such that AZD9574 and a lipophilic statin are administered following a complete or partial response to a platinum-based therapy or following at least one cycle of platinum-based therapy.

In an embodiment, AZD9574 is administered as a maintenance therapy in patients having recurrent cancer, in particular recurrent ovarian cancer, following a complete or partial response to platinum-based therapy. The lipophilic statin may also be administered in this regimen, such that AZD9574 and a lipophilic statin are administered in patients having recurrent cancer, in particular recurrent ovarian cancer, following a complete or partial response to a platinum-based therapy.

In another embodiment, AZD9574 is administered in patients, in particular patients having ovarian cancer, having had three or more prior chemotherapy treatments and whose cancer is HR deficient. In an embodiment, AZD9574 is administered in patients whose cancer is HR deficient in BRCA1 and/or BRCA2. The lipophilic statin may also be administered in this regimen, such that AZD9574 and a lipophilic statin are administered in patients, in particular patients having ovarian cancer, having had three or more prior chemotherapy treatments and whose cancer is HR deficient.

In an embodiment, AZD9574 and lipophilic statin is administered in patients characterised by having a cancer which is HR proficient.

In an embodiment, the PARP inhibitor and agent that is capable of modulating the cholesterol biosynthesis pathway, such as an LSS inhibitor, is administered before a patient receives platinum-based therapy for the treatment of cancer.

In an embodiment, the PARP inhibitor is administered as a maintenance therapy following a complete or partial response to a platinum-based therapy. In other words, the PARP inhibitor is administered following at least one cycle of platinum-based therapy. The agent that is capable of modulating the cholesterol biosynthesis pathway, such as an LSS inhibitor, may also be administered in this regimen, such that a PARP inhibitor and agent that is capable of modulating the cholesterol biosynthesis pathway are administered following a complete or partial response to a platinum-based therapy or following at least one cycle of platinum-based therapy.

In an embodiment, the PARP inhibitor is administered as a maintenance therapy in patients having recurrent cancer, in particular recurrent ovarian cancer, following a complete or partial response to platinum-based therapy. The agent that is capable of modulating the cholesterol biosynthesis pathway, such as an LSS inhibitor, may also be administered in this regimen, such that a PARP inhibitor and agent that is capable of modulating the cholesterol biosynthesis pathway are administered in patients having recurrent cancer, in particular recurrent ovarian cancer, following a complete or partial response to a platinum-based therapy.

In another embodiment, the PARP inhibitor is administered in patients, in particular patients having ovarian cancer, having had three or more prior chemotherapy treatments and whose cancer is associated with HRD positive status. The HRD positive status may be characterised by a deleterious or suspected deleterious BRCA mutation or in patients with genomic instability and who have progressed more than six months after response to the last platinum-based therapy. The agent that is capable of modulating the cholesterol biosynthesis pathway, such as an LSS inhibitor, may also be administered in this regimen, such that the PARP inhibitor and the agent that is capable of modulating the cholesterol biosynthesis pathway, such as an LSS inhibitor, is administered in patients, in particular patients having ovarian cancer, having had three or more prior chemotherapy treatments and whose cancer is associated with HRD positive status.

In an embodiment, the PARP inhibitor and agent that is capable of modulating the cholesterol biosynthesis pathway, such as an LSS inhibitor, is administered in patients characterised by having a cancer which is HR proficient.

In an embodiment, niraparib and agent that is capable of modulating the cholesterol biosynthesis pathway, such as an LSS inhibitor, is administered before a patient receives platinum-based therapy for the treatment of cancer.

In an embodiment, niraparib is administered as a maintenance therapy following a complete or partial response to a platinum-based therapy. In other words, niraparib is administered following at least one cycle of platinum-based therapy. The agent that is capable of modulating the cholesterol biosynthesis pathway, such as an LSS inhibitor, may also be administered in this regimen, such that niraparib and the agent that is capable of modulating the cholesterol biosynthesis pathway are administered following a complete or partial response to a platinum-based therapy or following at least one cycle of platinum-based therapy.

In an embodiment, niraparib is administered as a maintenance therapy in patients having recurrent cancer, in particular recurrent ovarian cancer, following a complete or partial response to platinum-based therapy. The agent that is capable of modulating the cholesterol biosynthesis pathway, such as an LSS inhibitor, may also be administered in this regimen, such that niraparib and the agent that is capable of modulating the cholesterol biosynthesis pathway are administered in patients having recurrent cancer, in particular recurrent ovarian cancer, following a complete or partial response to a platinum-based therapy.

In another embodiment, the niraparib is administered in patients, in particular patients having ovarian cancer, having had three or more prior chemotherapy treatments and whose cancer is associated with HRD positive status. The HRD positive status may be characterised by a deleterious or suspected deleterious BRCA mutation or in patients with genomic instability and who have progressed more than six months after response to the last platinum-based therapy. The agent that is capable of modulating the cholesterol biosynthesis pathway, such as an LSS inhibitor, may also be administered in this regimen, such that niraparib and the agent that is capable of modulating the cholesterol biosynthesis pathway, such as an LSS inhibitor, is administered in patients, in particular patients having ovarian cancer, having had three or more prior chemotherapy treatments and whose cancer is associated with HRD positive status.

In an embodiment, niraparib and the agent that is capable of modulating the cholesterol biosynthesis pathway, such as an LSS inhibitor, is administered in patients characterised by having a cancer which is HR proficient.

In an embodiment, olaparib is administered before a patient receives platinum-based therapy for the treatment of cancer.

In an embodiment, olaparib and the agent that is capable of modulating the cholesterol biosynthesis pathway is administered before a patient receives platinum-based therapy for the treatment of cancer.

In another embodiment, olaparib is administered as a maintenance therapy following a complete or partial response to a platinum-based therapy. In other words, olaparib is administered following at least one cycle of platinum-based therapy. The agent that is capable of modulating the cholesterol biosynthesis pathway may also be administered in this regimen, such that olaparib and the agent that is capable of modulating the cholesterol biosynthesis pathway are administered following a complete or partial response to a platinum-based therapy or following at least one cycle of platinum-based therapy.

In an embodiment, olaparib is administered as a maintenance therapy in patients having recurrent cancer, in particular recurrent ovarian cancer, following a complete or partial response to platinum-based therapy. The agent that is capable of modulating the cholesterol biosynthesis pathway may also be administered in this regimen, such that olaparib and the agent that is capable of modulating the cholesterol biosynthesis pathway are administered in patients having recurrent cancer, in particular recurrent ovarian cancer, following a complete or partial response to a platinum-based therapy.

In another embodiment, olaparib is administered in patients, in particular patients having ovarian cancer, having had three or more prior chemotherapy treatments and whose cancer is HR deficient. In an embodiment, olaparib is administered in patients whose cancer is HR deficient in BRCA1 and/or BRCA2. The agent that is capable of modulating the cholesterol biosynthesis pathway may also be administered in this regimen, such that olaparib and the agent that is capable of modulating the cholesterol biosynthesis pathway are administered in patients, in particular patients having ovarian cancer, having had three or more prior chemotherapy treatments and whose cancer is HR deficient.

In an embodiment, olaparib and the agent that is capable of modulating the cholesterol biosynthesis pathway is administered in patients characterised by having a cancer which is HR proficient.

In an embodiment, AZD5305 and the agent that is capable of modulating the cholesterol biosynthesis pathway is administered before a patient receives platinum-based therapy for the treatment of cancer.

In another embodiment, AZD5305 is administered as a maintenance therapy following a complete or partial response to a platinum-based therapy. In other words, AZD5305 is administered following at least one cycle of platinum-based therapy. The agent that is capable of modulating the cholesterol biosynthesis pathway may also be administered in this regimen, such that AZD5305 and a agent that is capable of modulating the cholesterol biosynthesis pathway are administered following a complete or partial response to a platinum-based therapy or following at least one cycle of platinum-based therapy.

In an embodiment, AZD5305 is administered as a maintenance therapy in patients having recurrent cancer, in particular recurrent ovarian cancer, following a complete or partial response to platinum-based therapy. The agent that is capable of modulating the cholesterol biosynthesis pathway may also be administered in this regimen, such that AZD5305 and the agent that is capable of modulating the cholesterol biosynthesis pathway are administered in patients having recurrent cancer, in particular recurrent ovarian cancer, following a complete or partial response to a platinum-based therapy.

In another embodiment, AZD5305 is administered in patients, in particular patients having ovarian cancer, having had three or more prior chemotherapy treatments and whose cancer is HR deficient. In an embodiment, AZD5305 is administered in patients whose cancer is HR deficient in BRCA1 and/or BRCA2. The agent that is capable of modulating the cholesterol biosynthesis pathway may also be administered in this regimen, such that AZD5305 and the agent that is capable of modulating the cholesterol biosynthesis pathway are administered in patients, in particular patients having ovarian cancer, having had three or more prior chemotherapy treatments and whose cancer is HR deficient.

In an embodiment, AZD5305 and the agent that is capable of modulating the cholesterol biosynthesis pathway is administered in patients characterised by having a cancer which is HR proficient.

In an embodiment, AZD9574 and the agent that is capable of modulating the cholesterol biosynthesis pathway is administered before a patient receives platinum-based therapy for the treatment of cancer.

In another embodiment, AZD9574 is administered as a maintenance therapy following a complete or partial response to a platinum-based therapy. In other words, AZD9574 is administered following at least one cycle of platinum-based therapy. The agent that is capable of modulating the cholesterol biosynthesis pathway may also be administered in this regimen, such that AZD9574 and a agent that is capable of modulating the cholesterol biosynthesis pathway are administered following a complete or partial response to a platinum-based therapy or following at least one cycle of platinum-based therapy.

In an embodiment, AZD9574 is administered as a maintenance therapy in patients having recurrent cancer, in particular recurrent ovarian cancer, following a complete or partial response to platinum-based therapy. The agent that is capable of modulating the cholesterol biosynthesis pathway may also be administered in this regimen, such that AZD9574 and the agent that is capable of modulating the cholesterol biosynthesis pathway are administered in patients having recurrent cancer, in particular recurrent ovarian cancer, following a complete or partial response to a platinum-based therapy.

In another embodiment, AZD9574 is administered in patients, in particular patients having ovarian cancer, having had three or more prior chemotherapy treatments and whose cancer is HR deficient. In an embodiment, AZD9574 is administered in patients whose cancer is HR deficient in BRCA1 and/or BRCA2. The agent that is capable of modulating the cholesterol biosynthesis pathway may also be administered in this regimen, such that AZD9574 and the agent that is capable of modulating the cholesterol biosynthesis pathway are administered in patients, in particular patients having ovarian cancer, having had three or more prior chemotherapy treatments and whose cancer is HR deficient.

In an embodiment, AZD9574 and the agent that is capable of modulating the cholesterol biosynthesis pathway is administered in patients characterised by having a cancer which is HR proficient.

In an embodiment, the invention relates to a method for switching a PARP inhibitor treatment regimen in a patient with cancer in need thereof from a treatment regimen comprising a PARP inhibitor alone to a treatment regimen comprising a PARP inhibitor and a lipophilic statin. In an embodiment, the PARP inhibitor used in the treatment regimen comprising PARP inhibitor and lipophilic statin may comprise the same PARP inhibitor or different to the treatment regimen with PARP inhibitor alone.

In an embodiment, the invention relates to a method for switching a patient on treatment for cancer with a PARP inhibitor (such as niraparib, olaparib, rucaparib, talazoparib or AZD5305) from a treatment regimen comprising a PARP inhibitor alone (such as niraparib, olaparib, rucaparib, talazoparib or AZD5305) to a treatment regimen comprising a PARP inhibitor (such as niraparib, olaparib, rucaparib, talazoparib or AZD5305) and a lipophilic statin. In an embodiment, the PARP inhibitor used in the treatment regimen comprising PARP inhibitor and lipophilic statin may comprise the same PARP inhibitor or different to the treatment regimen with PARP inhibitor alone. For example, disclosed is a method for switching a patient on treatment for cancer with a PARP inhibitor, such as olaparib, from a treatment regimen comprising olaparib alone to a regimen comprising niraparib and a lipophilic statin. Alternatively, contemplated is a method for switching a patient on treatment for cancer with olaparib alone to a regimen comprising olaparib and lipophilic statin. Another example contemplated is a method for switching a patient on treatment for cancer with a PARP inhibitor, such as niraparib, from a treatment regimen comprising niraparib alone to a regimen comprising olaparib and a lipophilic statin.

In an embodiment, the invention relates to a method for switching a patient on treatment for cancer with a PARP inhibitor (such as niraparib, olaparib, rucaparib, talazoparib, AZD9574 or AZD5305) from a treatment regimen comprising a PARP inhibitor alone (such as niraparib, olaparib, rucaparib, talazoparib, AZD9574 or AZD5305) to a treatment regimen comprising niraparib and a lipophilic statin.

In an embodiment, the invention relates to a method for switching a patient on treatment for cancer with a PARP inhibitor (such as niraparib, olaparib, rucaparib, talazoparib or AZD5305) from a treatment regimen comprising a PARP inhibitor alone (such as niraparib, olaparib, rucaparib, talazoparib or AZD5305) to a treatment regimen comprising niraparib and a lipophilic statin.

In an embodiment, the invention relates to a method for switching a patient on treatment for cancer with a PARP inhibitor (such as niraparib, olaparib, rucaparib, talazoparib, AZD9574 or AZD5305) from a treatment regimen comprising a PARP inhibitor alone (such as niraparib, olaparib, rucaparib, talazoparib or AZD5305) to a treatment regimen comprising a PARP inhibitor and a lipophilic statin.

In an embodiment, the invention relates to a method for switching a patient on treatment for cancer with niraparib from a treatment regimen comprising niraparib alone to a treatment regimen comprising niraparib and a lipophilic statin.

In an embodiment, the invention relates to a method of treating cancer in a patient in need thereof, said patient on treatment for said cancer comprising a PARP inhibitor and switching said patient to a treatment regimen comprising administering to the patient a therapeutically effective amount of a PARP inhibitor; and a therapeutically effective amount of a lipophilic statin.

In an embodiment, the invention relates to a method of treating cancer in a patient in need thereof, said patient on treatment for said cancer comprising a PARP inhibitor (such as niraparib, olaparib, rucaparib, talazoparib, AZD9574 or

AZD5305) and switching said patient to a treatment regimen comprising administering to the patient a therapeutically effective amount of niraparib and a therapeutically effective amount of a lipophilic statin.

In an embodiment, the invention relates to a method of treating cancer in a patient in need thereof, said patient on treatment for said cancer comprising a PARP inhibitor (such as niraparib, olaparib, rucaparib, talazoparib or AZD5305) and switching said patient to a treatment regimen comprising administering to the patient a therapeutically effective amount of niraparib and a therapeutically effective amount of a lipophilic statin.

In other words, the invention also relates to a method of treating cancer in a patient on treatment for said cancer with a PARP inhibitor and the patient is switched to be treated for said cancer by administering to the patient a therapeutically effective amount of a PARP inhibitor; and a therapeutically effective amount of a lipophilic statin.

In an embodiment, the invention also relates to a method of treating cancer in a patient on treatment for said cancer with a PARP inhibitor and the patient is switched to be treated for said cancer by administering to the patient a therapeutically effective amount of niraparib; and a therapeutically effective amount of a lipophilic statin.

Also disclosed herein is a method of treating cancer in a patient in need thereof, the method comprising administering to the patient (i) a therapeutically effective amount of a PARP inhibitor; and (ii) a therapeutically effective amount of a lipophilic statin, wherein the patient was previously on treatment with a PARP inhibitor alone, wherein the PARP inhibitor may be the same or different.

Also disclosed herein is a method of treating cancer in a patient in need thereof, the method comprising administering to the patient (i) a therapeutically effective amount of niraparib; and (ii) a therapeutically effective amount of a lipophilic statin, wherein the patient was previously on treatment with a PARP inhibitor alone, wherein the PARP inhibitor may be the same or different.

Also disclosed here is a method of treating cancer in a patient in need thereof, the method comprising administering to the patient (i) a therapeutically effective amount of niraparib; and (ii) a therapeutically effective amount of a lipophilic statin, wherein the patient was previously on treatment with niraparib alone.

The invention will now be described with reference to the following non-limiting examples.

### EXAMPLES

### Example 1

This example describes a post-hoc analysis of the PRIMA clinical trial.

### Statistical Analysis

This was an exploratory post hoc analysis.

For the subgroup analyses, we performed a two-sided log-rank test using randomization stratification factors (best response to last platinum; HRD status; and time from penultimate platinum to progression) to analyze PFS (progression free survival), which was summarized with the use of Kaplan-Meier methods. We estimated hazard ratios with two-sided 95% Confidence Intervals (CIs) using a stratified Cox proportional hazards (PH) model, with the stratification factors used in randomization.

Subgroup analyses were performed to assess the potential synergistic effect of statin and niraparib on PFS by testing the treatment by statin co-medication in a Cox proportional hazards model that also included niraparib treatment and statin co-medication treatment as covariates and the stratification factors used in randomization as strata, and niraparib-by-statin interaction terms were tested in the Cox proportional hazards model. The Cox model for interaction testing also included stratification factors used in randomization as covariates. Hazard ratios of niraparib statin interaction and their 95% CIs were estimated from Cox PH model including both niraparib treatment and statin treatment as covariates and the stratification factors used in randomization as strata.

### Results

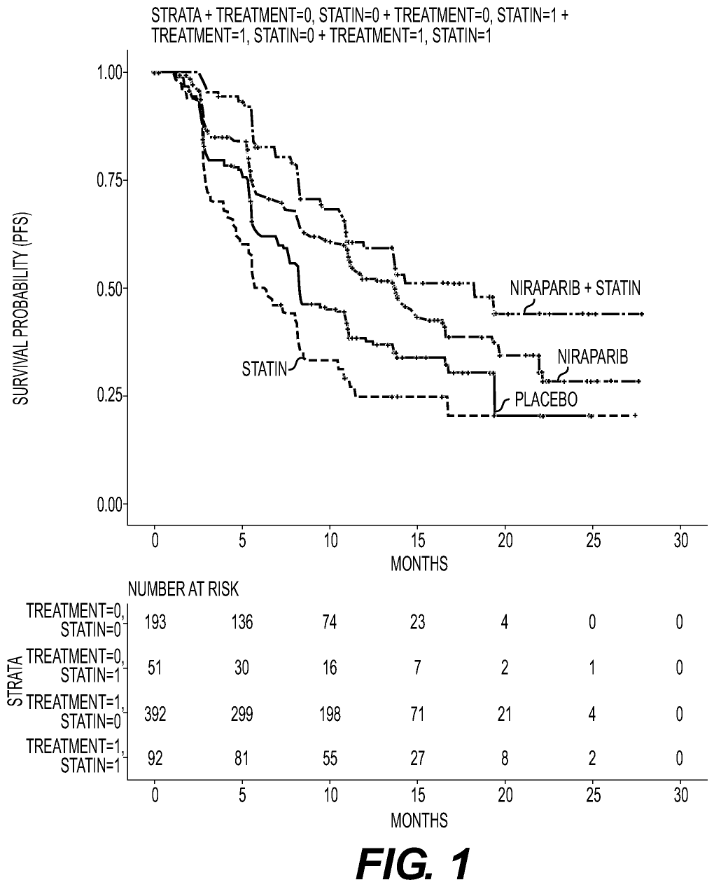

FIG. 1: all patients in PRIMA trial are categorized into four groups, niraparib+statin (N=92), niraparib (N=392), placebo (N=193), statin (N=51), and their survival curves are shown in FIG. 1. Interestingly, the hazard ratio for statin alone is 1.59, which suggests that the statin cohort has a worse outcome than the placebo cohort. Statin is a class of drug used to treat high cholesterol. Therefore, it is hypothesised that patients in the statin cohort are older and heavier. In other words, statin treatment correlates with a bad outcome. Hazard ratios of niraparib statin interaction is 0.49 and their 95% confidence intervals are (0.29, 0.81), which indicates that niraparib and statin interaction (combination) is statistically significant in this post hoc analysis.

The mPFS for study participants who were receiving treatment with niraparib and already on statin treatment was 18.2 months compared to an mPFS of 13.7 months for niraparib alone. In addition, the mPFS for study participants receiving statin treatment only was 6.0 months and the mPFS for study participants on placebo was 8.3 months.

In summary, it can be seen from FIG. 1 that subjects on statin treatment alone have a poor prognosis (even relative to those subjects on placebo in the PRIMA clinical trial). Subjects who receive statin therapy as well as a PARP inhibitor (in particular niraparib) have a much better prognosis relative to either niraparib or statin alone. For example, the mPFS following treatment of a subject having cancer with statin alone is 6.0 months compared to 18.2 months.

### Example 2

The objective of this study was to assess synergy in cancer cell killing between PARP inhibitors (specifically olaparib and niraparib) and statins (specifically atorvastatin, simvastatin, fluvastatin and pravastatin) in 16 cancer cell lines grown in 2D and covering 10 tumor indications. The following study gives an indication of whether synergy exists between a PARP inhibitor and a statin and is supportive of the post-hoc analysis from Example 1.

The cell lines used in the study were as follows in Table 1.

TABLE 1

| | Tumor Type | Name | HRD score* |
|---|---|---|---|
| 1 | Breast | MDA-MB-231 | 23 |
| 2 | | BT549 | 18 |
| 3 | Ovarian | SKOV3 | 17 |
| 4 | Liver | HepG2 | 8 |
| 5 | | SNU-423 | 16 |
| 6 | Lung | A549 | 19 |
| 7 | | NCI-H1437 | 33 |
| 8 | Pancreatic | CAPAN-2 | 16 |
| 9 | | BxPC-3 | 17 |
| 10 | Colon | HCT-116 | 14 |
| 11 | | LoVo | 4 |
| 12 | Head&neck | Cal 27 | 17 |
| 13 | | Detroit 562 | 33 |
| 14 | Gastric | KATO III | 16 |
| 15 | Kidney | 786-O | 11 |
| 16 | Skin | A375 | 29 |

### Summary of the Experimental Procedure

Cells were grown in RPMI 1640, 10% FBS, 2 mM L-alanyl-L-glutamine, 1 mM Na pyruvate, or an appropriate medium if required by a particular cell line. Cells were seeded into 384-well plates and incubated in a humidified atmosphere of 5% CO2 at 37° C. Compounds were added the day following cell seeding. After a 5-day incubation period, cells were fixed and stained with DAPI (4′,6-diamidino-2-phenylindole) to allow fluorescence imaging of nuclei.

All compounds besides staurosporine (a positive control for cell killing) were serially diluted in 3-fold steps from the highest test concentration (specified in Table 2), and assayed over 9 concentrations with a maximum assay concentration of 0.2% DMSO (dimethyl sulfoxide). Staurosporine was diluted in half-log (3.16-fold) steps and assayed over 10 concentrations with a maximum assay concentration of 0.1% DMSO.

Automated fluorescence microscopy was carried out using a Molecular Devices ImageXpress Micro XL high-content imager, and images were collected with a 4X objective. 16-bit TIFF (Tag Image File Format) images were acquired and analyzed with MetaXpress 5.1.0.41 software. Curve-fitting, calculations, and report generation was performed by Eurofins using a custom data reduction engine and MathIQ based software (AIM).

TABLE 2

| | | Pre- | | | |
|---|---|---|---|---|---|
| | | weighted | | | |
| | | dry | | Final | Max final |
| Compound | | compound | Stock | volume of | test conc. |
| Information | M.W. | (mg) | conc. (mM) | stock (μL) | (μM) |
| Niraparib | — | — | 30 | 600 | 30 |
| Olaparib | — | — | 30 | 600 | 30 |
| Atorvastatin | — | — | 10 | 600 | 10 |
| Simvastatin | — | — | 10 | 600 | 10 |
| Pravastatin | — | — | 10 | 600 | 10 |
| Fluvastatin | — | — | 10 | 600 | 10 |
| Staurosporine | 466.5 | 1.0 | 1 | 2144 | 1 |

The data set contained calculated Excess over Bliss independence (Bliss score) using the following equation:

$$Bliss Score = C - ( ( A + B ) - A B )$$

Where A and B are the fractional growth inhibitions of two drugs added alone at a given dose, and C is the observed inhibition when the same drugs are added in combination.

BlissScoreMean values for each concentration mix were derived from the curveFitValues (the % of control value of the fitted curve obtained at a given individual or pair of concentrations).

To better assess the results, the plate BlissScore was subsequently calculated using the provided curveFitValues and the open software: SynergyFinder 2.0 Lanevski, A., Giri, K. A., Aittokallio, T., 2020. SynergyFinder 2.0: visual analytics of multi-drug combination synergies. Nucleic Acids Research. gkaa216, doi.org/10.1093/nar/qkaa216). The calculated BlissScorevalues were in agreement with the provided BlissScoreMean results. Additionally, the SynergyFinder 2.0 created 2D and 3D synergy maps highlighting the synergistic and antagonistic dose regions in red and green colors, respectively.

The 2D and 3D maps were used to perform a quality control of the data and classify the findings into synergy groups.

Previous work has indicated high variability between technical and biological replicates of different viability assays (Colony Formation Assay, CellFluor/Live Cell Protease and CellTiterGlow readouts, internal GSK data) performed to assess synergy between PARP inhibitors and statins in 2D cancer cell lines. This suggests that those assays as well as the biological model might not be sensitive enough to robustly asses the synergistic effects of both drug classes. In order to cover the potentially low sensitivity of the readout and enable better identification of conditions where synergy can be observed, we included two types of synergy classification: synergy score and synergy region.

Synergy score=BlissScore for the whole plate (concentration matrix) BSₘₐₜᵣᵢₓ>2 and synergy detected in one region covering at least 2 neighbouring concentrations of compound A and 2 neighbouring concentration of compound B.

Synergy region=BlissScore for the whole plate (concentration matrix) BSₘₐₜᵣᵢₓ<2 but a significant region of synergy detected which fulfills both of the following criteria:
1. one area of at least 4 concentrations at which BlissScore is positive covering at least 2 concentrations of compound A and 2 concentration of compound B
2. at least one combination of compound A & compound B in this area having a BS_{AxBy}>10 (BSₘₐₓ). This value has been reported by the authors of SynergyFinder 2.0 as a good indication that the interaction between two drugs is likely to be synergistic.

Classes of synergy characterised as follows:

### Results

A summary of the data observed using the above analysis is as follows:

The data output showed region of synergy for a combination of niraparib and lipophilic statin (fluvastatin, simvastatin or atorvastatin) as well as olaparib and lipophilic statin (fluvastatin, simvastatin or atorvastatin) in several HRP cancer cell lines. No synergy was observed between niraparib or olaparib and hydrophilic pravastatin in tested cell lines.

### Example 3

The objective of this study was to assess synergy in cancer cell killing between PARP inhibitors (specifically niraparib) and statins (specifically atorvastatin, simvastatin, fluvastatin) in HRD cancer cell line UWB1.289 grown in 2D. The following study gives an indication of whether synergy exists between a PARP inhibitor and a statin in HRD cell line and is supportive of the post-hoc analysis from Example 1.

### Summary of the Experimental Procedure

Cells were grown in medium as described by ATCC (50% RPMI 1640, 50% MEGM, 3% FBS, without gentamycin-amphotericin component of SingleQuot additives). Cells were seeded into 96-well plates (2.000 cells per well) and incubated in a humidified atmosphere of 5% CO2 at 37° C. Compounds were added the day following cell seeding. Triplicates (3 plates) were performed per tested condition. After a 7-day incubation period, cells viability has been measured using CellTiterGlo reagent (ATP levels) using the Perkin Elmer plate reader Envision 2105 and the corresponding analysis software Envision manager 1.14.3049.528.

All compounds were serially diluted in 4-fold steps from the highest test concentration (specified in Table 3), and assayed over 6 concentrations with a maximum assay concentration of 0.2% DMSO.

TABLE 3

| Compound | Stock | Max final test |
|---|---|---|
| Information | conc. (mM) | conc. (μM) |
| Niraparib | 100 | 25 |
| Atorvastatin | 50 | 10 |
| Simvastatin | 50 | 10 |
| Fluvastatin | 50 | 10 |

To assess the results, the plate BlissScore was calculated using viability results (% of control) and the open software: SynergyFinder 2.0 as described in Example 2. The same criteria for data analysis and interpretation has been applied.

### Results

The data output showed synergy for a combination of niraparib and fluvastatin in ovarian HRD cancer cell line UWB1.289.

### Example 4

The objective of this study was to assess synergy in cancer cell killing between PARP inhibitors (specifically AZD5305, niraparib, olaparib) and statins (specifically lipophilic fluvastatin) in HRD ovarian cancer cell line UWB1.289 grown in 2D. The following study gives an indication of whether synergy also exists between a new PARP inhibitor AZD5305 and a statin in HRD background.

### Summary of the Experimental Procedure

Cells were grown in medium as described by ATCC (50% RPMI 1640, 50% MEGM, 3% FBS, without gentamycin-amphotericin component of SingleQuot additives). Cells were seeded into 96-well plates (2.000 cells per well) and incubated in a humidified atmosphere of 5% CO2 at 37° C. Compounds were added the day following cell seeding. Triplicates (3 plates) were performed per tested condition. After a 7-day incubation period, cells viability has been measured using CellTiterGlo reagent (ATP levels) using the Perkin Elmer plate reader Envision 2105 and the corresponding analysis software Envision manager 1.14.3049.528.

All compounds were serially diluted in 2- or 4-fold steps from the highest test concentration (specified in Table 4) and assayed over 6 concentrations with a maximum assay concentration of 0.2% DMSO. Final concentrations of PARP inhibitor (AZD5305) in experiment 1:0.39 μM, 0.097 μM, 0.024 μM, 0.0061M, 0.0015UM, 0.00039 μM (in some experiments: 0.012 μM instead 0.39 μM was used). Final concentrations of niraparib and olaparib: 3.125 μM, 1.56 μM, 0.78 μM, 0.39 μM, 0.097 μM, 0.024 μM (in some experiments: 0.195 μM instead 3.125 μM was used). Final concentrations of fluvastatin: 1.25 μM, 0.625 μM, 0.3125 μM, 0.156 μM, 0.039 μM, 0.097 μM.

TABLE 4

| Compound | Stock | Max final test |
|---|---|---|
| Information | conc. (mM) | conc. (μM) |
| AZD5305 | 50 | 0.39 or 0.097 |
| olaparib | 50 | 3.125 or 1.56 |
| niraparib | 50 | 3.125 or 1.56 |
| Fluvastatin | 50 | 1.25 |

To assess the results, the plate Bliss Score was calculated using viability results (% of control) and the open software: SynergyFinder 2.0 as described in Example 2. The same criteria for data analysis and interpretation has been applied. Classes of synergy characterised as follows:

### Results

The data output showed Bliss Score (BS) synergy (or at least synergy region) for all tested PARP inhibitors (AZD5305, olaparib, niraparib) and fluvastatin in HRD ovarian cancer cell line UWB1.289.

### Example 5

The objective of this study was to assess synergy in cancer cell killing between PARP inhibitors (specifically niraparib, olaparib and AZD5305) and statins (specifically lipophilic atorvastatin, hydrophilic pravastatin) in HRP triple negative breast cancer (TNBC) cell line MDA-MB-231 grown in 2D. The following study gives an indication of whether synergy also exists between PARP inhibitors and a statin in HRP background.

### Summary of the Experimental Procedure

MDA-MB-231 cells were grown in RPMI-based medium (RPMI 1640, 1 mM sodium pyruvate, 2 mM Glutamax, 10% FBS). Cells were seeded into 96-well plates (2.000 cells) and incubated in a humidified atmosphere of 5% CO2 at 37° C. Compounds were added the day following cell seeding. Triplicates or quadruplicates (3 or 4 plates) were performed per tested condition. After a 7-day incubation period, cells viability has been measured using CellTiterGlo reagent (ATP levels) using the Perkin Elmer plate reader Envision 2105 and the corresponding analysis software Envision manager 1.14.3049.528.

All compounds were serially diluted in 2- or 4-fold steps from the highest test concentration (specified in Table 5) and assayed over 6 concentrations with a maximum assay concentration of 0.2% DMSO. Final concentrations of PARP inhibitors (niraparib, olaparib and AZD5305): 25 μM, 12.5 μM, 6.25 μM, 1.56 μM, 0.39 μM, 0.097 μM (in some experiments 0.024 μM were used instead of 12.5 μM). Final concentrations of atorvastatin and pravastatin: 5 μM, 2.5 μM, 1.25 μM, 0.625 μM, 0.156 μM, 0.039 μM (in some experiments 10 μM were used instead of 5 μM).

TABLE 5

| Compound | Stock | Max final test |
|---|---|---|
| Information | conc. (mM) | conc. (μM) |
| Niraparib | 50 | 25 |
| Olaparib | 50 | 25 |
| AZD5305 | 50 | 25 |
| Atrovastatin | 50 | 5 or 10 |
| Pravastatin | 50 | 5 or 10 |

To assess the results, the plate Bliss Score (BS) was calculated using viability results (% of control) and the open software: SynergyFinder 2.0 as described in Example 2. The same criteria for data analysis and interpretation has been applied.

Classes of synergy characterised as follows:

### Results

In breast HRP cancer cell line MDA-MB-231 (TNBC), the data output showed reproducibly high Bliss Score (BS) synergy for a combination of all three PARP inhibitors (niraparib, olaparib and AZD5305) with lipophilic atorvastatin. No synergy score or synergy region was detected for a combination of niraparib or AZD5305 and pravastatin (hydrophilic statin).

### Example 6

The objective of this study was to assess synergy in cancer cell killing between PARP inhibitors (specifically niraparib, olaparib) and statins (specifically lipophilic atorvastatin and fluvastatin) in HRP breast cancer cell line HCC1954 (TNBC) grown in 2D. The following study gives an indication of whether synergy exists between PARP inhibitors and a statin in HRP background.

### Summary of the Experimental Procedure

HCC1954 cells were grown in RPMI-based medium (RPMI 1640, 2 mM Glutamax, 5% FBS). Cells were seeded into 96-well plates (2.000 cells per well) and incubated in a humidified atmosphere of 5% CO2 at 37° C. Compounds were added the day following cell seeding. Triplicates (3 plates) were performed per tested condition. After a 7-day incubation period, cells viability has been measured using CellTiterGlo reagent (ATP levels) using the Perkin Elmer plate reader Envision 2105 and the corresponding analysis software Envision manager 1.14.3049.528.

All compounds were serially diluted in 2- or 4-fold steps from the highest test concentration (specified in Table 6) and assayed over 6 concentrations with a maximum assay concentration of 0.2% DMSO. Final concentrations of niraparib: 6.25 μM, 3.125 μM, 1.56 μM, 0.78 μM, 0.39 μM, 0.097 μM. Final concentrations of olaparib: 12.5 μM, 6.25 μM, 3.125 μM, 1.56 μM, 0.39 μM, 0.097 μM. Final concentrations of Atrovastatin: 10 μM, 5 μM 2.5 μM, 1.25 μM, 0.625 μM, 0.156 μM. Final concentrations of Fluvastatin: 5 μM, 2.5 μM, 1.25 μM, 0.625 μM, 0.156 μM, 0.039 μM.

TABLE 6

| | Stock | Max final |
|---|---|---|
| Compound | conc. | test conc. |
| Information | (mM) | (μM) |
| Niraparib | 50 | 25 |
| Olaparib | 50 | 25 |
| Atrovastatin | 50 | 10 |
| Fluvastatin | 50 | 5 or 2.5 |

To assess the results, the plate Bliss Score (BS) was calculated using viability results (% of control) and the open software: SynergyFinder 2.0 as described in Example 2. The same criteria for data analysis and interpretation has been applied.

Classes of synergy characterised as follows:

### Results

In breast HRP cancer cell line HCC1954, the data output showed reproducibly Bliss Score (BS) synergy for a combination of both PARP inhibitors (niraparib, olaparib) with lipophilic fluvastatin, as well as niraparib with lipophilic atorvastatin.

### Example 7

The objective of this study was to assess synergy in tumor cell killing between PARP inhibitors (specifically niraparib) and statins (specifically lipophilic atorvastatin and hydrophilic pravastatin) in Patient-Derived Ovarian Cancer Organoids (HUB) grown in 3D. This organoid line has a monoallelic mutation in BRCA1 and was regarded as HR proficient (HRP) based on CHORD values from De Witte C J, et al Cell Reports 2020. The following study gives an indication of whether synergy also exists between PARP inhibitor niraparib and a statin in primary tumor cells growing in organoid form.

### Summary of the Experimental Procedure

Organoids were grown in organoid tumour culture medium containing matrigel and were seeded into 384-well plates (200 organoids per well, size range 20-70 um) and incubated in a humidified atmosphere of 5% CO2 at 37° C. for 1 hr before addition of compounds. Five replicate plates were prepared containing the tested conditions. After a 7-day incubation period, cell viability was measured using CellTiter-Glo 3D reagent (Promega G9682). Luminescence (ATP levels) was measured on a PHERAstar plate reader using the MARS application software and the resulting data analysed in TIBCO Spotfire software.

All compounds were serially diluted in 3-fold steps from the highest test concentration (specified in Table 7) and assayed over 7 concentrations with a maximum assay concentration of 0.5% DMSO. Final concentrations of niraparib: 30 mM, 10 mM, 3.33 mM, 1.11 mM, 0.37 mM, 0.12 mM, 0.041 mM. Final concentrations of atorvastatin: 5 mM, 1.6667 mM, 0.55 mM, 0.185 mM, 0.0617 mM, 0.020 mM, 0.0069 mM. Final concentrations of pravastatin: 50 mM, 16.667 mM, 5.5 mM, 1.85 mM, 0.617 mM, 0.20 mM, 0.069 mM.

TABLE 7

| Compound | Stock | Max final test |
|---|---|---|
| Information | conc. (mM) | conc. (μM) |
| niraparib | 12 | 30 |
| atorvastatin | 2 | 5 |
| pravastatin | 20 | 50 |

To assess the results, the “Bliss Interaction Index I” was calculated using viability results (% of inhibition) and Bliss Independence model (according to Zhao et al. 2014, A New Bliss Independence Model to Analyze Drug Combination Data). Generated output consists of Contour Plot (I>0 indicates synergism and I<0 antagonism) and 95% Confidence Interval Lower Limits (Values>0 indicate statistically significant synergism. In addition, same data set was analyzed using the open software: SynergyFinder 2.0 as described in Example 2.

### Results

The data output showed that with both methods statistically significant and biologically relevant synergism could be detected for niraparib-atorvastatin combination in Patient-Derived Ovarian Cancer Organoids (HUB). No synergy/synergy region (as described in example 2) was observed between niraparib and hydrophilic pravastatin.

### Example 8

The objective of this study was to assess the effect of the combination between the steroid biosynthesis pathway inhibitors (specifically Lanosterol Synthase (LSS) inhibitor: Ro 48-8071) and PARP inhibitors (specifically olaparib and niraparib) on the viability of HRP triple negative breast cancer (TNBC) cell line HCC70 grown in 2D. The following study gives an indication of whether also other inhibitors of the cholesterol biosynthesis pathway (beyond lipophilic statins) in combination with PARP inhibitors can increase efficiency of cancer cell killing in HRP background.

### Summary of the Experimental Procedure

Cells were grown in standard medium (RPMI 1640, 2 mM Glutamax, 5% FBS). Cells were seeded into 96-well plates (3.000 cells per well) and incubated in a humidified atmosphere of 5% CO2 at 37° C. Compounds were added the day following cell seeding. Triplicates or quadruplicates (3 or 4 plates) were performed per tested condition. After a 7-day incubation period, cells viability has been measured using CellTiterGlo reagent (ATP levels) using the Perkin Elmer plate reader Envision 2105 and the corresponding analysis software Envision manager 1.14.3049.528.

Both PARP inhibitors has been tested at a final concentration of 6.25 μM as single agent or in combination with LSS inhibitor Ro 48-8071 (final concentration 300 nM), see Table 8.

TABLE 8

| Compound | Stock conc. | Final test |
|---|---|---|
| Information | (mM) | conc. (μM) |
| Niraparib | 50 | 6.25 |
| Olaparib | 50 | 6.25 |
| Ro 48-8071 | 10 | 0.3 |

To assess the results, viability results (% of control) were plotted as mean from 4 biological replicates (each biological replicate consisting of 3-4 technical replicates). Significance was tested using unpaired t test with Welch's correction.

### Results

In breast HRP cancer cell line HCC70, the data output showed that addition of LSS inhibitor Ro 48-8071 improves olaparib and niraparib efficiency in reducing tumor cell viability.

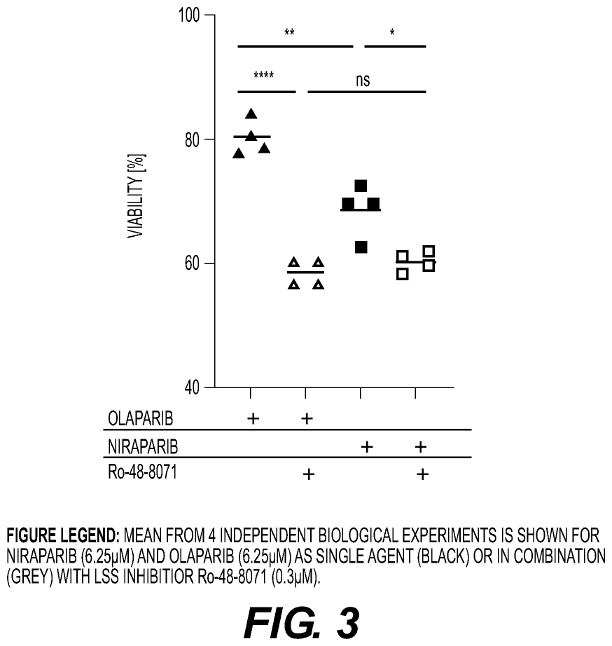

The results can be seen in FIG. 3.

1. A method of treating cancer in a patient in need thereof, the method comprising administering to the patient
(i) a therapeutically effective amount of a poly (ADP-ribose) polymerase inhibitor (PARP inhibitor); and
(ii) a therapeutically effective amount of a lipophilic statin.

2. A method of treating cancer in a patient in need thereof, the method comprising administering to the patient a therapeutically effective amount of a poly (ADP-ribose) polymerase inhibitor (PARP inhibitor) selected from the group consisting of niraparib, olaparib, talazoparib, rucaparib, veliparib and AZD5305 or a pharmaceutically acceptable salt thereof, wherein the patient has been previously treated with a therapeutically effective amount of a lipophilic statin.

3. The method according to claim 1, wherein the PARP inhibitor is selected from the group consisting of niraparib, olaparib, talazoparib, rucaparib, veliparib and AZD5305 or a pharmaceutically acceptable salt thereof.

4. The method according to claim 3, wherein the PARP inhibitor is selected from the group consisting of niraparib, olaparib, talazoparib and rucaparib or a pharmaceutically acceptable salt thereof.

5. The method according to claim 4, wherein the PARP inhibitor is niraparib or a pharmaceutically acceptable salt thereof.

6. The method according to claim 1, wherein the PARP inhibitor is niraparib tosylate monohydrate.

7. The method according to claim 5, wherein the dose of niraparib is 100 mg, 200 mg or 300 mg taken once daily.

8. The method according to claim 1, wherein the lipophilic statin has a Log D>0 at a pH of 7.0.

9. The method according to claim 1, wherein the lipophilic statin is selected from the group consisting of atorvastatin, simvastatin, fluvastatin, lovastatin, pitavastatin and cerivastatin.

10. The method according to claim 9, wherein the lipophilic statin is selected from the group consisting of atorvastatin, simvastatin and fluvastatin.

11. The method according to claim 10, wherein the lipophilic statin is atorvastatin or simvastatin.

12. The method according to claim 1, wherein the cancer is selected from the group consisting of breast, colorectal, gastric, head & neck, kidney, liver, lung, ovarian, pancreatic, skin, sarcoma, brain, endometrial, thyroid, neuroendocrine, bladder, cervical, melanoma and prostate cancer.

13. The method according to claim 12, wherein the cancer is selected from breast, colorectal, gastric, head & neck, kidney, liver, lung, ovarian, pancreatic and skin cancer.

14. The method according to claim 13, wherein the cancer is selected from the group consisting of breast, ovarian and lung cancer.

15. The method according to claim 1, wherein at least the PARP inhibitor is administered as a maintenance therapy following complete or partial response to at least one platinum-based therapy.

16. The method according to claim 1, wherein at least the PARP inhibitor is administered before platinum-based therapy.

17. The method according to claim 1, wherein the patient is characterised by having a cancer which is HR proficient.

18. The method according to claim 1, wherein the patient is characterised by having a cancer which is HR deficient.

19. The method according to claim 18, wherein the patient is characterised by having a deleterious or suspected deleterious mutation in BRCA1 and/or BRCA2.

20-25. (canceled)

26. The method according to claim 2, wherein the lipophilic statin is selected from the group consisting of atorvastatin, simvastatin, fluvastatin, lovastatin, pitavastatin and cerivastatin.
